(19) United States
(12) Patent Application Publication
(10) Pub. No.: US 2024/0145031 A1
(43) Pub. Date: May 2, 2024

(54) Methods and devices for drug repositioning and drug repurposing as nuclear receptor modulators

(71) Applicant: Precision Medicine Lab, Vught (CA)

(72) Inventors: Renè Houtman, Culemborg (NL); Tim Kievits, 's-Hertogenbosch (NL)

(73) Assignee: Precision Medicine Lab, Vught (NL)

(21) Appl. No.: 18/480,098

(22) Filed: Oct. 3, 2023

Related U.S. Application Data
(60) Provisional application No. 63/378,695, filed on Oct. 7, 2022.

(51) Int. Cl. G16B 15/30

(52) U.S. Cl. CPC G16B 15/30

(57) ABSTRACT

The invention relates to methods and devices for drug repositioning and drug repurposing as nuclear receptor modulators based on nuclear receptor cofactor testing.

### BACKGROUND OF THE INVENTION

Nuclear receptors are ligand-inducible transcriptional factors that control multiple biological phenomena, including proliferation, differentiation, reproduction, metabolism, and the maintenance of homeostasis. Members of the nuclear receptor superfamily have marked structural and functional similarities, and their domain functionalities and regulatory mechanisms have been well studied. Various modulators of nuclear receptors, including agonists and antagonists, have been developed as drug candidates or lead compounds. Many assay systems are currently available to evaluate the modulation of nuclear receptor functions and are useful as screening tools in the discovery and development of new modulators.ⁱ Forty-eight types of nuclear receptors have been found in humans, and 25 of these are orphan receptors with unknown endogenous ligandsⁱⁱ. The ligand-dependent action of nuclear receptors includes that the activated receptors translocate into the nucleus, bind to the specific sites of DNA, and regulate the target gene expression to elicit various events, such as cell proliferation, differentiation, reproduction, metabolism, and the maintenance of homeostasis^{iii iv}. Some orphan receptors are known to act constitutively as transcription-promoting factors and to play roles in releasing transcriptional repression^{v}.

Nuclear receptors have evolved from a single gene and have been systematically, classified based on their structural and functional domains as shown in FIG. 1 describing the structure of the Estrogen Receptor alpha^{vi}. The A/B region contains a transcription-promoting region (activation function 1; AF-1). The C region located near the center of the receptor contains a zinc (Zn)-finger structure and includes the DNA-binding domain (DBD) responsible for recognition and binding to a specific DNA sequence^{vii}. The D region, known as the hinge region, contains the nuclear localization signal (NLS). The E region includes the ligand-binding domain (LBD) and has a ligand-dependent transcriptional activation function (activation function 2; AF-2). The F region is an optional C-terminal domain Steroid receptors such as the estrogen receptor (ER) (ERα: NR3A1, ERβ: NR3A2) and androgen receptor (AR: NR3C4) bind to DNA as homodimers. The retinoid. X receptor (RXR) (RXRα: NR2B1, RXRβ: NR2B2, RXRγ: NR2B3) forms heterodimers with various receptors as partners to bind to their specific DNA sites, Some receptors bind to DNA as monomers, including some orphan receptors^{viii ix}. The two AF regions regulate transcription by directly interacting with transcription cofactors, such as corepressors and coactivators^{x xi xii}.

Protein-protein interactions involving transcriptional co-regulators, corepressors, and coactivators, play an important role in the regulation of nuclear receptor functions. Therefore, Protein-protein interactions can also be targets for drug discovery, with both peptides^{xiii} and chemical compounds^{xiv xv} having the potential to disrupt these Protein-protein interactions.

Chemical compounds which can interact the protein-protein interactions, such as nuclear receptors, can be derived from several sources. One such source are chemicals derived from natural products libraries. This is because natural product libraries contain a diversity of biological species. The physiological activities and chemical structures of natural compounds also show immense diversity, and many of them have proved useful as pharmaceuticals or leads for drug discovery. To date, more than 200,000 natural compounds have been isolated from various biological resources, and their structures have been elucidatec^{xvi}.

Another source of chemical compounds which can interact with protein-protein interactions are libraries of known compounds. For instance, the NHRCore Library is a computationally selected library of more than 2,700 small molecules included in ChemBridge's CORE Library^{xvii}. Compounds in this library were selected for synthesis based on similarity to 3D pharmacophore fingerprints generated from published compounds with activity against nuclear hormone receptors. More than 250 novel scaffolds designed by ChemBridge are represented in the NHRCore Library.

Another source of chemical compounds which can interact with protein-protein interactions are libraries of known compounds already used as therapeutic agents for diseases other than the original target diseases, i.e., so-called “drug repositioning” or “drug repurposing”^{xvii}. Drug repositioning or drug repurposing has certain advantages over traditional drug discovery which is a complex, time-consuming, tedious, costly, and risky process. There are various methods or steps involved in the drug discovery process which include random screening, molecular manipulation, computational strategy, drug metabolic study, and serendipitous research. New drug candidates are developed mainly through different stages including identification of disease state or target, target validation, identification of lead, lead optimization, preclinical study, toxicity study, formulation, clinical trial, approval process, and marketing of the drugs with post-marketing surveillance or safety monitoring, etc. As, these processes are laborious, time-consuming, and expensive with a high risk of failure, drug repurposing is considered a novel approach to identify new therapeutic purposes for existing or already marketed or approved drugs by EMA, MHRA, or for instance FDA. Drug repurposing is also represented as drug repositioning, drug reprofiling, drug rescuing, drug recycling, and therapeutic switching. Drug repositioning involves the investigation of known drugs for new therapeutic indications. For example, antiviral drugs like Remdesivir, and Favipiravir, and antimalarial drugs like Chloroquine, Hydroxychloroquine are repositioning for the treatment of COVID-19. It is estimated that 10-12 years are required for the development of new drug molecules in the traditional drug discovery approach. While the estimated time is between 1 and 3 years in the case of the drug repositioning method. The average expenditure required to obtain a new pharmacologically active drug to market is USD 1.24 billion by traditional drug development process^{xviii}.

There are various drugs that have been repurposed and are approved or under clinical trials including all-trans retinoic acid, Amphotericin, Aspirin, Atomoxetine, Auranofin, Celecoxib, Celebrex, Dapoxetine, Dapsone, Digoxin, Duloxetine, Eflornithine, Fingolimod, Fosmidomycin, Fumagillin, Itraconazol, Itraconazol, Ketoconazole, Lomitapide, Metformin, Minocycline, Minoxidil, MSDC-0160, Nitroxoline, Paromomycin, Pentostatin, Pioglitazone, Raloxifene, Rapamycin, Riluzole, Rituximab, Sildenafil, Sodium nitrite, Tamoxifen and Thalidomide, Topiramate, Zidovudine^{xix xx}. One other library that contains drugs which could be used for a repurposing or repositioning program is the Library of Pharmacologically Active Compounds tLOPAC®1280) which contains 1280 bioactive small molecules.

In recent years the development of successful nuclear receptor modulators has proven challenging as for instance seen in the field of glucocorticoid receptor modulator development which has shown promising initial results such as LGD-5552, AL-438, MK-5932, GW870086, BI650348, Mapracorat, Dagracorat, AZD5423, AZD7594 and AZD9567 but these compounds never got past the pre-clinical stage or failed later in clinical trials^{xxi}. It is well-known that the success rate for the development of any kind of small molecule drug from bench to clinic is very low, typically starting from 10,000 compounds to end up with one market-approved drug^{xxii}. Current tools for screening potential selective nuclear receptor modulators such as modulators of the glucocorticoid receptor suffer from shortcomings and do not always capture the complexity of nuclear receptor signaling. A nuclear receptor may be allosterically regulated through interactions with its corresponding response elements and cofactors^{xxiii} which should be considered when testing potential novel nuclear receptor modulators.

Cofactors are regulatory proteins that bind to transcription factors, such as nuclear receptors to assist in their transcriptional activity. Depending on various factors, e.g., coregulator availability, posttranslational modifications, and characteristics of the ligand, a specific set of coregulators is recruited to the receptor. Knowledge about these interactions is essential for the understanding and prediction of nuclear receptor-mediated effects in different systems, as these coregulators influence the transcriptional outcome of nuclear receptors. One method to identify the effect of ligands or drug molecules such as glucocorticoid receptor modulators on the nuclear receptor-coregulator interactions is using microarrays^{xxiv}. Such a microarray technology described in the art consists of several coregulator-derived peptides spotted onto the microarray. Each amino acid peptide contains a nuclear receptor binding motif, including the LXXLL or LXXXIXXXL sequence, derived from known coactivator or corepressor proteins, respectively.

### SUMMARY OF THE INVENTION

The invention is based on the idea of testing known drugs in a drug repurposing test, as potential nuclear receptor modulators using a nuclear receptor cofactor recruitment-based profiling system and using a drug repurposing computing device. This drug repurposing computing device includes a computing environment including storage and a software algorithm that allows weighted cofactor recruitment data of repurposing or repositioning drugs including safe drugs to be compared with known regulatory approved and or clinically failed drugs to reposition or repurpose the drugs for one or more nuclear receptors.

The invention thus utilizes a nuclear receptor cofactor recruitment screening assay and a drug repurposing computing device to seek known marketed drugs as potential nuclear receptor modulators which can be used to treat human diseases wherein the disease is mediated through alterations of nuclear receptor activity.

Drug repurposing strategy finds new uses other than the original medical indications of existing drugs. Finding new indications for such drugs will benefit patients who require a potential new therapy sooner since known drugs have acceptable safety and pharmacokinetic profiles.

In one embodiment, the methods or uses as taught herein comprise: selecting, by a drug repurposing computing device, a set of at least one regulatory approved drug and at least one negative control drug and optionally at least one failed drug of which said regulatory approved drugs are known to interact with the ligand binding domain of a nuclear receptor said interaction leading to a change in the affinity of said nuclear receptor for binding to a set of at least two peptides derived from cofactor proteins generating a cofactor binding ratio value for each peptide in the presence and absence of said regulatory approved drug and said negative control drugs and optionally said failed drugs targeting said nuclear receptor thereby generating a cofactor binding ratio signature for the collection of tested cofactor peptides from said tested regulatory approved drugs and said negative control drugs and optionally said set of failed drugs and; associating, by the drug repurposing computing device, the cofactor binding ratio signature using weighted cofactor binding ratio values with the regulatory approved drugs targeting said nuclear receptor; calculating, by the drug repurposing computing device, a probability score of a drug to be repurposed for the selected nuclear receptor based on its measured cofactor binding ratio signature in comparison to said weighted cofactor binding ratio signature from said regulatory approved drugs and said negative control drug and optionally said failed drug.

In certain embodiments, said nuclear receptor is a full-length nuclear receptor.

In certain embodiments, said full-length nuclear receptor is derived from a cell lysate.

In yet another embodiment, the methods or uses as taught herein generate said probability score by said drug repurposing computing device wherein at least two nuclear receptors are used.

In certain embodiments, said nuclear receptors are selected from the list: NR0A1, NR0B1, NR0B2, NR1A1, NR1A2, NR1B1, NR1B2, NR1B3, NR1C1, NR1C2, NR1C3, NR1D1, NR1D2, NR1E1, NR1F1, NR1F2, NR1F3, NR1G1, NR1H1, NR1H2, NR1H3, NR1H4, NR1H5, NR1I1, NR1I2, NR1I3, NR1J1, NR1J2, NR1J3, NR1K1, NR2A1, NR2A2, NR2B1, NR2B2, NR2B3, NR2B4, NR2C1, NR2C2, NR2E1, NR2E3, NR2F1, NR2F2, NR2F6, NR3A1, NR3A2, NR3B1, NR3B2, NR3B3, NR3C1, NR3C2, NR3C3, NR3C4, NR3D, NR3E, NR3F, NR4A1, NR4A2, NR4A3, NR5A1, NR5A2, NR5B1, NR6A1, NR7A1, NR7B1, NR7C1, and NR8A1.

In certain embodiments said nuclear receptors described herein comprise wild-type nuclear receptor and one or more mutant nuclear receptors.

In yet another embodiment the methods or uses as taught herein describe wild-type nuclear receptor variant and one or more nuclear receptor variants each containing one or more post-translational modifications such as acetylation, methylation, phosphorylation, SUMOylation, and ubiquitination.

### BRIEF DESCRIPTION OF THE FIGURES

FIG. 1: Summary of the functional domains of nuclear receptors, and the post-translational modification sites in ERα: NR3A1; A: acetylation, M: methylation, P: phosphorylation, S: SUMOylation, U: ubiquitination.

FIG. 2: An example binding profile in a bar chart for coregulator peptide no 22 with name NCOA1_1421_1444 for each of the tested repurposing compounds of table 5 and the positive control (PC), cortisol, and the negative control (NC), DMSO.

FIG. 3: 4 panels one for each of the tested repurposing compounds of table 5 showing the binding profiles as bars for a set of 20 selected cofactor peptides (table 2). The binding profile of the positive control, cortisol, is shown as a grey line which is the same in each of the 4 panels.

FIG. 4: The log fold change of binding for coregulator peptide no 22 with name NCOA1_1421_1444 versus the binding to the positive control, cortisol, for each of the tested repurposing drugs is shown. A grey area is used to show the log fold change of binding range between −0.5 log and 0.5 log. The top dashed line shows the log fold change of binding for the positive control, cortisol.

FIG. 5: 4 panels, one for each of the tested repurposing compounds showing the log fold change of binding for a set of 20 selected cofactor peptides (table 2) shown as dotted data points. The log fold change of binding for the positive control, cortisol is shown as a line which is the same in each of the panels. A grey area is used to show the log fold change of binding range between −0.5 log and 0.5 log. The top dashed line shows the log fold change of binding for the positive control, cortisol.

FIG. 6: 20 panels, one for each of the tested cofactor peptides showing the log fold change of binding for the tested repurposing compounds of table 5 as dotted data points. A grey area is used to indicate the area where the log fold change of binding is between −0.25 log and 0.25 log.

### BRIEF DESCRIPTION OF THE TABLES

TABLE 1: Table consisting of amino-acid sequences of 70 cofactor peptides used in Example 1. The table further consisting of the identification number of the cofactor peptide (ID), the name of the cofactor peptide (Peptide_Name), the amino-acid sequence of the cofactor peptides (Peptide_Sequence), the Universal Protein—Uniprot—identification which is based on a central repository of protein data created by combining the Swiss-Prot, TrEMBL and PIR-PSD databases (Uniprot_Accession) and lastly the unique gene identification number (GeneID).

TABLE 2: Table consisting of a subset of 20 cofactor peptides selected from the set of 70 cofactor peptides of table 1

TABLE 3: Table consisting of a subset of 14 cofactor peptides selected from the set of 70 cofactor peptides based on an assigned weight (outlined in table 7) having a minimum absolute log fold change on the positive control, cortisol, of 0.5. These peptides are called “Weight-A cofactor peptides used in table 10”.

TABLE 4: Table consisting of a subset of 4 cofactor peptides selected from the set of 70 cofactor peptides as described in example 4. These peptides are called “Weight-B cofactor peptides used in table 10”.

TABLE 5: The Names and Cas Numbers of the tested repurposing compounds used in the examples and the positive control; cortisol are outlined in this table.

TABLE 6: The table displays log fold change results of example 1 of the tested drugs outlined in table 5 for the list of cofactor peptides of table 1. No value is shown in case the log fold change is below the threshold of example 1.

TABLE 7: The table displays the 8 weight factors used in example 3 for each of the 8 absolute log fold change ranges. In essence for every increase of the absolute log fold change by 0.25 the weight factor is doubled to a maximum of 1 at the highest log fold change range. The first two log fold change ranges up to an absolute log fold change of 0.5 have a weight factor of 0.

TABLE 8: The table displays the log fold change results of example 3 of the tested drugs outlined in table 5 for the list of the cofactor peptides of table 3 which are selected based on the weight factors of table 7. No value is shown in case the log fold change is below the threshold of example 3.

TABLE 9: The table displays log fold change results of example 4 of the tested drugs outlined in table 5 for the list of the cofactor peptides of table 4. No value is shown in case the log fold change is below the threshold of example 4.

TABLE 10: The similarities between the tested repositioning drugs of table 5 versus cortisol using the coefficient of determination, R2, between the log fold change data for the tested repurposing drug versus the positive control, cortisol is shown using either the table 1 (No weight), table 3 (Weight-A) or table 4 (Weight B) set of cofactor peptides.

### DETAILED DESCRIPTION

All scientific and technical terms used in this application have meanings commonly used in the art unless otherwise specified. As used in this application, the following words or phrases have the meanings specified.

The terms “a” and “an” refer to one or more than one (i.e., to at least one) of the grammatical object of the article. The term “or” refers to “and/or” unless explicitly indicated to refer to alternatives only or unless the alternatives are mutually exclusive. The term “subject” includes living organisms such as humans, monkeys, cows, sheep, horses, pigs, cattle, goats, dogs, cats, mice, rats, cultured cells, and transgenic species thereof. In a preferred embodiment, the subject is a human.

The term “administering” includes routes of administration that allow the active ingredient of the invention to perform its intended function. The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition. The term “prevent,” “prevention” or “preventing” means inhibition or averting of symptoms associated with the target disease.

The phrase “therapeutically effective amount” refers to the amount of a compound, material, or composition comprising a compound of the present invention, which is effective for producing a desired therapeutic effect, at a reasonable benefit/risk ratio applicable to any medical treatment. Reference throughout this specification to “one embodiment”, “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may be referring to the same embodiment. Furthermore, the features, structures, or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention and form different embodiments, as would be understood by those in the art. For example, in the appended claims, any of the claimed embodiments can be used in any combination.

Throughout this disclosure, various publications, patents, and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. Through further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

The term “failed drug” refers to safe drug candidates that failed in clinical trials and which were shelved as drug developers channeled resources to the next candidates in the pipeline. Specialized companies are emerging with new methods and a fresh point of view to assist pharmaceutical developers in turning failed drug candidates from one therapeutic area into successful treatments in another.

The term “negative control compound” refers to drugs that do not bind to the nuclear receptors used in the nuclear receptor cofactor recruitment screening assay.

The term “drug repositioning” and “drug repurposing” refers to drugs or compounds, preferably organic compounds, with a size comparable to those organic molecules generally used in pharmaceuticals. Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. Said preferred small molecules include FDA approved small molecules as described in the Orange Book including: ABACAVIR SULFATE, ABALOPARATIDE, ABAMETAPIR, ABARELIX, ABEMACICLIB, ABIRATERONE ACETATE, ABROCITINIB, ACALABRUTINIB, ACAMPROSATE CALCIUM, ACARBOSE, ACEBUTOLOL HYDROCHLORIDE, ACETAMINOPHEN, ACETAZOLAMIDE, ACETAZOLAMIDE SODIUM, ACETIC ACID, ACETOHEXAMIDE, ACETOHYDROXAMIC ACID, ACETOPHENAZINE MALEATE, ACETRIZOATE SODIUM, ACETYLCHOLINE CHLORIDE, ACETYLCYSTEINE, ACETYLDIGITOXIN, ACITRETIN, ACLIDINIUM BROMIDE, ACRISORCIN, ACRIVASTINE, ACYCLOVIR, ACYCLOVIR SODIUM, ADAPALENE, ADEFOVIR DIPIVOXIL, ADENOSINE, AFAMELANOTIDE, AFATINIB DIMALEATE, AIR POLYMER-TYPE A, ALATROFLOXACIN MESYLATE, ALBENDAZOLE, ALBUMIN HUMAN, ALBUTEROL, ALBUTEROL SULFATE, ALCAFTADINE, ALCLOMETASONE DIPROPIONATE, ALCOHOL, ALECTINIB HYDROCHLORIDE, ALENDRONATE SODIUM, ALFENTANIL HYDROCHLORIDE, ALFUZOSIN HYDROCHLORIDE, ALISKIREN HEMIFUMARATE, ALITRETINOIN, ALKAVERVIR, ALLOPURINOL, ALLOPURINOL SODIUM, ALMOTRIPTAN MALATE, ALOGLIPTIN BENZOATE, ALOSETRON HYDROCHLORIDE, ALPELISIB, ALPHA-TOCOPHEROL ACETATE, ALPRAZOLAM, ALPROSTADIL, ALSEROXYLON, ALTRETAMINE, ALUMINUM HYDROXIDE, ALVIMOPAN, AMANTADINE HYDROCHLORIDE, AMBENONIUM CHLORIDE, AMBRISENTAN, AMCINONIDE, AMDINOCILLIN, AMIFAMPRIDINE PHOSPHATE, AMIFOSTINE, AMIKACIN SULFATE, AMILORIDE HYDROCHLORIDE, AMINO ACIDS, AMINOCAPROIC ACID, AMINOGLUTETHIMIDE, AMINOHIPPURATE SODIUM, AMINOLEVULINIC ACID HYDROCHLORIDE, AMINOPHYLLINE, AMINOSALICYLATE SODIUM, AMINOSALICYLIC ACID, AMINOSALICYLIC ACID RESIN COMPLEX, AMIODARONE HYDROCHLORIDE, AMISULPRIDE, AMITRIPTYLINE HYDROCHLORIDE, AMLEXANOX, AMLODIPINE BENZOATE, AMLODIPINE BESYLATE, AMLODIPINE MALEATE, AMMONIA N-13, AMMONIUM CHLORIDE, AMMONIUM LACTATE, AMODIAQUINE HYDROCHLORIDE, AMOXAPINE, AMOXICILLIN, AMPHETAMINE, AMPHETAMINE ADIPATE, AMPHETAMINE ASPARTATE, AMPHETAMINE ASPARTATE, AMPHETAMINE RESIN COMPLEX, AMPHETAMINE SULFATE, AMPHOTERICIN B, AMPICILLIN SODIUM, AMPICILLIN/AMPICILLIN TRIHYDRATE, AMPRENAVIR, ANAGRELIDE HYDROCHLORIDE, ANASTROZOLE, ANGIOTENSIN II ACETATE, ANIDULAFUNGIN, ANILERIDINE HYDROCHLORIDE, ANILERIDINE PHOSPHATE, ANISINDIONE, ANISOTROPINE METHYLBROMIDE, ANTAZOLINE PHOSPHATE, APALUTAMIDE, APIXABAN, APOMORPHINE HYDROCHLORIDE, APRACLONIDINE HYDROCHLORIDE, APREMILAST, APREPITANT, ARBUTAMINE HYDROCHLORIDE, ARDEPARIN SODIUM, ARFORMOTEROL TARTRATE, ARGATROBAN, ARGININE HYDROCHLORIDE, ARIPIPRAZOLE, ARIPIPRAZOLE LAUROXIL, ARMODAFINIL, ARSENIC TRIOXIDE, ARTEMETHER, ARTESUNATE, ARTICAINE HYDROCHLORIDE, ASCIMINIB HYDROCHLORIDE, ASCORBIC ACID, ASENAPINE, ASENAPINE MALEATE, ASPIRIN, ATAZANAVIR SULFATE, ATENOLOL, ATOGEPANT, ATOMOXETINE HYDROCHLORIDE, ATORVASTATIN CALCIUM, ATOVAQUONE, ATRACURIUM BESYLATE, ATROPINE, ATROPINE SULFATE, AURANOFIN, AVACOPAN, AVANAFIL, AVAPRITINIB, AVATROMBOPAG MALEATE, AVIBACTAM SODIUM, AVOBENZONE, AXITINIB, AZACITIDINE, AZATADINE MALEATE, AZATHIOPRINE, AZATHIOPRINE SODIUM, AZELAIC ACID, AZELASTINE HYDROCHLORIDE, AZILSARTAN KAMEDOXOMIL, AZITHROMYCIN, AZITHROMYCIN DIHYDRATE, AZLOCILLIN SODIUM, AZTREONAM, BACAMPICILLIN HYDROCHLORIDE, BACITRACIN, BACITRACIN ZINC, BACLOFEN, BALOXAVIR MARBOXIL, BALSALAZIDE DISODIUM, BARICITINIB, BARIUM SULFATE, BAZEDOXIFENE ACETATE, BECLOMETHASONE DIPROPIONATE, BECLOMETHASONE DIPROPIONATE MONOHYDRATE, BEDAQUILINE FUMARATE, BELINOSTAT, BELUMOSUDIL MESYLATE, BELZUTIFAN, BEMPEDOIC ACID, BENAZEPRIL HYDROCHLORIDE, BENDAMUSTINE HYDROCHLORIDE, BENDROFLUMETHIAZIDE, BENOXINATE HYDROCHLORIDE, BENTIROMIDE, BENTOQUATAM, BENZNIDAZOLE, BENZONATATE, BENZOYL PEROXIDE, BENZPHETAMINE HYDROCHLORIDE, BENZQUINAMIDE HYDROCHLORIDE, BENZTHIAZIDE, BENZTROPINE MESYLATE, BENZYL ALCOHOL, BENZYL BENZOATE, BEPOTASTINE BESILATE, BEPRIDIL HYDROCHLORIDE, BEROTRALSTAT HYDROCHLORIDE, BESIFLOXACIN HYDROCHLORIDE, BETA CAROTENE, BETAINE, BETAMETHASONE, BETAMETHASONE ACETATE, BETAMETHASONE BENZOATE, BETAMETHASONE DIPROPIONATE, BETAMETHASONE SODIUM PHOSPHATE, BETAMETHASONE VALERATE, BETAXOLOL HYDROCHLORIDE, BETAZOLE HYDROCHLORIDE, BETHANECHOL CHLORIDE, BETHANIDINE SULFATE, BETRIXABAN, BEXAROTENE, BICALUTAMIDE, BICTEGRAVIR SODIUM, BIMATOPROST, BINIMETINIB, BIPERIDEN HYDROCHLORIDE, BIPERIDEN LACTATE, BISACODYL, BISMUTH SUBCITRATE POTASSIUM, BISMUTH SUBSALICYLATE, BISOPROLOL FUMARATE, BITOLTEROL MESYLATE, BIVALIRUDIN, BLEOMYCIN SULFATE, BOCEPREVIR, BORTEZOMIB, BOSENTAN, BOSUTINIB MONOHYDRATE, BREMELANOTIDE ACETATE, BRETYLIUM TOSYLATE, BREXANOLONE, BREXPIPRAZOLE, BRIGATINIB, BRILLIANT BLUE G, BRIMONIDINE TARTRATE, BRINCIDOFOVIR, BRINZOLAMIDE, BRIVARACETAM, BROMFENAC SODIUM, BROMOCRIPTINE MESYLATE, BROMODIPHENHYDRAMINE HYDROCHLORIDE, BROMPHENIRAMINE MALEATE, BUCLIZINE HYDROCHLORIDE, BUDESONIDE, BUMETANIDE, BUPIVACAINE, BUPIVACAINE HYDROCHLORIDE, BUPRENORPHINE, BUPRENORPHINE HYDROCHLORIDE, BUPROPION HYDROBROMIDE, BUPROPION HYDROCHLORIDE, BUSPIRONE HYDROCHLORIDE, BUSULFAN, BUTABARBITAL SODIUM, BUTENAFINE HYDROCHLORIDE, BUTOCONAZOLE NITRATE, BUTORPHANOL TARTRATE, CAB AZITAXEL, CABERGOLINE, CABOTEGRAVIR, CABOTEGRAVIR SODIUM, CABOZANTINIB S-MALATE, CAFFEINE CITRATE, CAFFEINE, CALCIFEDIOL, CALCIPOTRIENE, CALCITONIN HUMAN, CALCITONIN SALMON, CALCITONIN SALMON RECOMBINANT, CALCITRIOL, CALCIUM ACETATE, CALCIUM CARBONATE, CALCIUM CHLORIDE, CALCIUM GLUCEPTATE, CALCIUM GLUCONATE, CALCIUM METRIZOATE, CALCIUM OXYBATE, CALCIUM, CANAGLIFLOZIN, CANDESARTAN CILEXETIL, CANDICIDIN, CANGRELOR, CANNABIDIOL, CAPECITABINE, CAPMATINIB HYDROCHLORIDE, CAPREOMYCIN SULFATE, CAPSAICIN, CAPTOPRIL, CARBACHOL, CARBAMAZEPINE, CARBENICILLIN DISODIUM, CARBENICILLIN INDANYL SODIUM, CARBIDOPA, CARBINOXAMINE MALEATE, CARBOPLATIN, CARBOPROST TROMETHAMINE, CARFILZOMIB, CARGLUMIC ACID, CARIPRAZINE HYDROCHLORIDE, CARISOPRODOL, CARMUSTINE, CARPHENAZINE MALEATE, CARPROFEN, CARTEOLOL HYDROCHLORIDE, CARVEDILOL, CARVEDILOL PHOSPHATE, CASIMERSEN, CASPOFUNGIN ACETATE, CEDAZURIDINE, CEFACLOR, CEFADROXIL/CEFADROXIL HEMIHYDRATE, CEFAMANDOLE NAFATE, CEFAZOLIN SODIUM, CEFDINIR, CEFDITOREN PIVOXIL, CEFEPIME HYDROCHLORIDE, CEFIDEROCOL SULFATE TOSYLATE, CEFIXIME, CEFMENOXIME HYDROCHLORIDE, CEFMETAZOLE SODIUM, CEFONICID SODIUM, CEFOPERAZONE SODIUM, CEFORANIDE, CEFOTAXIME SODIUM, CEFOTETAN DISODIUM, CEFOTIAM HYDROCHLORIDE, CEFOXITIN SODIUM, CEFPIRAMIDE SODIUM, CEFPODOXIME PROXETIL, CEFPROZIL, CEFTAROLINE FOSAMIL, CEFTAZIDIME, CEFTAZIDIME SODIUM, CEFTIBUTEN DIHYDRATE, CEFTIZOXIME SODIUM, CEFTOLOZANE SULFATE, CEFTRIAXONE SODIUM, CEFUROXIME AXETIL, CEFUROXIME SODIUM, CELECOXIB, CELLULOSE SODIUM PHOSPHATE, CENOBAMATE, CEPHALEXIN, CEPHALEXIN HYDROCHLORIDE, CEPHALOGLYCIN, CEPHALOTHIN SODIUM, CEPHAPIRIN SODIUM, CEPHRADINE, CERITINIB, CERIVASTATIN SODIUM, CERULETIDE DIETHYLAMINE, CETIRIZINE HYDROCHLORIDE, CETRORELIX, CETYL ALCOHOL, CEVIMELINE HYDROCHLORIDE, CHENODIOL, CHLOPHEDIANOL HYDROCHLORIDE, CHLORAMBUCIL, CHLORAMPHENICOL, CHLORAMPHENICOL SODIUM SUCCINATE, CHLORDIAZEPDXIDE, CHLORDIAZEPDXIDE HYDROCHLORIDE, CHLORHEXIDINE GLUCONATE, CHLORMERODRIN HG-197, CHLORMEZANONE, CHLOROPROCAINE HYDROCHLORIDE, CHLOROQUINE HYDROCHLORIDE, CHLOROQUINE PHOSPHATE, CHLOROTHIAZIDE, CHLOROTHIAZIDE SODIUM, CHLOROTRIANISENE, CHLOROXINE, CHLORPHENESIN CARBAMATE, CHLORPHENIRAMINE MALEATE, CHLORPHENIRAMINE POLISTIREX, CHLORPHENTERMINE HYDROCHLORIDE, CHLORPROMAZINE, CHLORPROMAZINE HYDROCHLORIDE, CHLORPROPAMIDE, CHLORPROTHIXENE, CHLORTETRACYCLINE HYDROCHLORIDE, CHLORTHALIDONE, CHLORZOXAZONE, CHOLESTYRAMINE, CHOLIC ACID, CHOLINE C-11, CHOLINE FENOFIBRATE, CHROMIC CHLORIDE, CHROMIC PHOSPHATE P-32, CICLESONIDE, CICLOPIROX, CIDOFOVIR, CILASTATIN SODIUM, CILOSTAZOL, CIMETIDINE, CIMETIDINE HYDROCHLORIDE, CINACALCET HYDROCHLORIDE, CINOXACIN, CIPROFLOXACIN, CIPROFLOXACIN HYDROCHLORIDE, CISAPRIDE MONOHYDRATE, CISATRACURIUM BESYLATE, CISPLATIN, CITALOPRAM HYDROBROMIDE, CITRIC ACID, CLADRIBINE, CLARITHROMYCIN, CLASCOTERONE, CLAVULANATE POTASSIUM, CLEMASTINE FUMARATE, CLEVIDIPINE, CLIDINIUM BROMIDE, CLINDAMYCIN HYDROCHLORIDE, CLINDAMYCIN PALMITATE HYDROCHLORIDE, CLINDAMYCIN PHOSPHATE, CLIOQUINOL, CLOBAZAM, CLOBETASOL PROPIONATE, CLOCORTOLONE PIVALATE, CLOFARABINE, CLOFAZIMINE, CLOFIBRATE, CLOMIPHENE CITRATE, CLOMIPRAMINE HYDROCHLORIDE, CLONAZEPAM, CLONIDINE, CLONIDINE HYDROCHLORIDE, CLOPIDOGREL BISULFATE, CLORAZEPATE DIPOTASSIUM, CLOTRIMAZOLE, CLOXACILLIN SODIUM, CLOZAPINE, COBICISTAT, COBIMETINIB FUMARATE, COCAINE HYDROCHLORIDE, CODEINE PHOSPHATE, CODEINE SULFATE, COLCHICINE, COLESEVELAM HYDROCHLORIDE, COLESTIPOL HYDROCHLORIDE, COLISTIMETHATE SODIUM, COLISTIN SULFATE, CONIVAPTAN HYDROCHLORIDE, COPANLISIB DIHYDROCHLORIDE, COPPER, COPPER DOTATATE CU-64, CORTICOTROPIN, CORTICOTROPIN-ZINC HYDROXIDE, CORTISONE ACETATE, COSYNTROPIN, CRISABOROLE, CRIZOTINIB, CROFELEMER, CROMOLYN SODIUM, CROTAMITON, CRYPTENAMINE ACETATES, CRYPTENAMINE TANNATES, CUPRIC CHLORIDE, CUPRIC SULFATE, CYANOCOBALAMIN, CYCLACILLIN, CYCLIZINE LACTATE, CYCLOBENZAPRINE HYDROCHLORIDE, CYCLOPENTOLATE HYDROCHLORIDE, CYCLOPHOSPHAMIDE, CYCLOSERINE, CYCLOSPORINE, CYCLOTHIAZIDE, CYCRIMINE HYDROCHLORIDE, CYPROHEPTADINE HYDROCHLORIDE, CYSTEAMINE BITARTRATE, CYSTEAMINE HYDROCHLORIDE, CYSTEINE HYDROCHLORIDE, CYTARABINE, DABIGATRAN ETEXILATE MESYLATE, DABRAFENIB MESYLATE, DACARBAZINE, DACLATASVIR DIHYDROCHLORIDE, DACOMITINIB, DACTINOMYCIN, DALBAVANCIN HYDROCHLORIDE, DALFAMPRIDINE, DALFOPRISTIN, DALTEPARIN SODIUM, DANAPAROID SODIUM, DANAZOL, DANTROLENE SODIUM, DAPAGLIFLOZIN, DAPIPRAZOLE HYDROCHLORIDE, DAPSONE, DAPTOMYCIN, DARIDOREXANT HYDROCHLORIDE, DARIFENACIN HYDROBROMIDE, DAROLUTAMIDE, DARUNAVIR, DASABUVIR SODIUM, DASATINIB, DASIGLUCAGON HYDROCHLORIDE, DAUNORUBICIN CITRATE, DAUNORUBICIN HYDROCHLORIDE, DECAMETHONIUM BROMIDE, DECITABINE, DEFERASIROX, DEFERIPRONE, DEFEROXAMINE MESYLATE, DEFIBROTIDE SODIUM, DEFLAZACORT, DEGARELIX ACETATE, DELAFLOXACIN MEGLUMINE, DELAVIRDINE MESYLATE, DEMECARIUM BROMIDE, DEMECLOCYCLINE HYDROCHLORIDE, DEOXYCHOLIC ACID, DESERPIDINE, DESFLURANE, DESIPRAMINE HYDROCHLORIDE, DESLANOSIDE, DESLORATADINE, DESMOPRESSIN ACETATE, DESOGESTREL, DESONIDE, DESOXIMETASONE, DESOXYCORTICOSTERONE ACETATE, DESOXYCORTICOSTERONE PIVALATE, DESVENLAFAXINE, DESVENLAFAXINE FUMARATE, DESVENLAFAXINE SUCCINATE, DEUTETRABENAZINE, DEXAMETHAS ONE, DEXAMETHASONE ACETATE, DEXAMETHASONE SODIUM PHOSPHATE, DEXBROMPHENIRAMINE MALEATE, DEXCHLORPHENIRAMINE MALEATE, DEXLANSOPRAZOLE, DEXMEDETOMIDINE HYDROCHLORIDE, DEXMETHYLPHENIDATE HYDROCHLORIDE, DEXRAZOXANE HYDROCHLORIDE, DEXTROAMPHETAMINE, DEXTROAMPHETAMINE SULFATE, DEXTROMETHORPHAN HYDROBROMIDE, DEXTROMETHORPHAN POLISTIREX, DEXTROSE, DEXTROTHYROXINE SODIUM, DEZOCINE, DIATRIZOATE MEGLUMINE, DIATRIZOATE SODIUM, DIAZEPAM, DIAZOXIDE, DIBUCAINE HYDROCHLORIDE, DICHLORPHENAMIDE, DICLOFENAC, DICLOFENAC EPOLAMINE, DICLOFENAC POTASSIUM, DICLOFENAC SODIUM, DICLOXACILLIN SODIUM, DICUMAROL, DICYCLOMINE HYDROCHLORIDE, DIDANOSINE, DIENESTROL, DIENOGEST, DIETHYLCARBAMAZINE CITRATE, DIETHYLPROPION HYDROCHLORIDE, DIETHYLSTILBESTROL, DIETHYLSTILBESTROL DIPHOSPHATE, DIFELIKEFALIN ACETATE, DIFLORASONE DIACETATE, DIFLUNISAL, DIFLUPREDNATE, DIGITOXIN, DIGOXIN, DIHYDROERGOTAMINE MESYLATE, DILTIAZEM HYDROCHLORIDE, DILTIAZEM MALATE, DIMENHYDRINATE, DIMERCAPROL, DIMETHYL FUMARATE, DIMETHYL SULFOXIDE, DIMYRISTOYL LECITHIN, DINOPROST TROMETHAMINE, DINOPROSTONE, DIPHEMANIL METHYLSULFATE, DIPHENHYDRAMINE CITRATE, DIPHENHYDRAMINE HYDROCHLORIDE, DIPHENIDOL HYDROCHLORIDE, DIPHENYLPYRALINE HYDROCHLORIDE, DIPIVEFRIN HYDROCHLORIDE, DIPYRIDAMOLE, DIRITHROMYCIN, DIROXIMEL FUMARATE, DISOPYRAMIDE PHOSPHATE, DISULFIRAM, DIVALPROEX SODIUM, DOBUTAMINE HYDROCHLORIDE, DOCETAXEL, DOCOSANOL, DOFETILIDE, DOLASETRON MESYLATE, DOLUTEGRAVIR SODIUM, DONEPEZIL HYDROCHLORIDE, DOPAMINE HYDROCHLORIDE, DORAVIRINE, DORIPENEM, DORZOLAMIDE HYDROCHLORIDE, DOXACURIUM CHLORIDE, DOXAPRAM HYDROCHLORIDE, DOXAZOSIN MESYLATE, DOXEPIN HYDROCHLORIDE, DOXERCALCIFEROL, DOXORUBICIN HYDROCHLORIDE, DOXYCYCLINE, DOXYCYCLINE CALCIUM, DOXYCYCLINE HYCLATE, DOXYLAMINE SUCCINATE, DROMOSTANOLONE PROPIONATE, DRONABINOL, DRONEDARONE HYDROCHLORIDE, DROPERIDOL, DROSPIRENONE, DROXIDOPA, DULOXETINE HYDROCHLORIDE, DUTASTERIDE, DUVELISIB, DYCLONINE HYDROCHLORIDE, DYDROGESTERONE, DYPHYLLINE, ECHOTHIOPHATE IODIDE, ECONAZOLE NITRATE, EDARAVONE, EDETATE CALCIUM DISODIUM, EDOXABAN TOSYLATE, EDROPHONIUM CHLORIDE, EFAVIRENZ, EFINACONAZOLE, EFLORNITHINE HYDROCHLORIDE, ELAGOLIX SODIUM, ELBASVIR, ELETRIPTAN HYDROBROMIDE, ELEXACAFTOR, ELIGLUSTAT TARTRATE, ELTROMBOPAG OLAMINE, ELUXADOLINE, ELVITEGRAVIR, EMEDASTINE DIFUMARATE, EMPAGLIFLOZIN, EMTRICITABINE, ENALAPRIL MALEATE, ENALAPRILAT, ENASIDENIB MESYLATE, ENCORAFENIB, ENFLURANE, ENFUVIRTIDE, ENOXACIN, ENOXAPARIN SODIUM, ENTACAPONE, ENTECAVIR, ENTRECTINIB, ENZALUTAMIDE, EPHEDRINE HYDROCHLORIDE, EPHEDRINE SULFATE, EPINASTINE HYDROCHLORIDE, EPINEPHRINE, EPINEPHRINE BITARTRATE, EPIRUBICIN HYDROCHLORIDE, EPLERENONE, EPOPROSTENOL SODIUM, EPROSARTAN MESYLATE, EPTIFIBATIDE, ERAVACYCLINE DIHYDROCHLORIDE, ERDAFITINIB, ERGOCALCIFEROL, ERGOLOID MESYLATES, ERGOTAMINE TARTRATE, ERIBULIN MESYLATE, ERLOTINIB HYDROCHLORIDE, ERTAPENEM SODIUM, ERTUGLIFLOZIN, ERYTHROMYCIN, ERYTHROMYCIN ESTOLATE, ERYTHROMYCIN ETHYLSUCCINATE, ERYTHROMYCIN GLUCEPTATE, ERYTHROMYCIN LACTOBIONATE, ERYTHROMYCIN STEARATE, ESCITALOPRAM OXALATE, ESKETAMINE HYDROCHLORIDE, ESLICARBAZEPINE ACETATE, ESMOLOL HYDROCHLORIDE, ESOMEPRAZOLE MAGNESIUM, ESOMEPRAZOLE SODIUM, ESOMEPRAZOLE STRONTIUM, ESTAZOLAM, ESTRADIOL, ESTRADIOL ACETATE, ESTRADIOL CYPIONATE, ESTRADIOL HEMIHYDRATE, ESTRADIOL VALERATE, ESTRAMUSTINE PHOSPHATE SODIUM, ESTROGENS, ESTRONE, ESTROPIPATE, ESZOPICLONE, ETELCALCETIDE, ETEPLIRSEN, ETHACRYNATE SODIUM, ETHACRYNIC ACID, ETHAMBUTOL HYDROCHLORIDE, ETHANOLAMINE OLEATE, ETHCHLORVYNOL, ETHINAMATE, ETHINYL ESTRADIOL, ETHIODIZED OIL, ETHIONAMIDE, ETHOPROPAZINE HYDROCHLORIDE, ETHOSUXIMIDE, ETHOTOIN, ETHOXZOLAMIDE, ETHYLESTRENOL, ETHYNODIOL DIACETATE, ETIDOCAINE HYDROCHLORIDE, ETIDRONATE DISODIUM, ETODOLAC, ETOMIDATE, ETONOGESTREL, ETOPOSIDE, ETOPOSIDE PHOSPHATE, ETRAVIRINE, ETRETINATE, EVANS BLUE, EVEROLIMUS, EXEMESTANE, EXENATIDE SYNTHETIC, EZETIMIBE, EZOGABINE, FAMCICLOVIR, FAMOTIDINE, FEBUXOSTAT, FEDRATINIB HYDROCHLORIDE, FELBAMATE, FELODIPINE, FENFLURAMINE HYDROCHLORIDE, FENOFIBRATE, FENOFIBRIC ACID, FENOLDOPAM MESYLATE, FENOPROFEN CALCIUM, FENTANYL, FENTANYL CITRATE, FENTANYL HYDROCHLORIDE, FERRIC AMMONIUM CITRATE, FERRIC CARBOXYMALTOSE, FERRIC CITRATE, FERRIC DERISOMALTOSE, FERRIC HEXACYANOFERRATE(II), FERRIC MALTOL, FERRIC OXYHYDROXIDE, FERRIC PYROPHOSPHATE CITRATE, FERROUS CITRATE, FERROUS SULFATE, FERUMOXIDES, FERUMOXSIL, FERUMOXYTOL, FESOTERODINE FUMARATE, FEXINIDAZOLE, FEXOFENADINE HYDROCHLORIDE, FIDAXOMICIN, FINAFLOXACIN, FINASTERIDE, FINERENONE, FINGOLIMOD HYDROCHLORIDE, FINGOLIMOD LAURYL SULFATE, FISH OIL TRIGLYCERIDES, FISH OIL, FLAVOXATE HYDROCHLORIDE, FLECAINIDE ACETATE, FLIBANSERIN, FLORBETABEN F-18, FLORBETAPIR F-18, FLORTAUCIPIR F-18, FLOXURIDINE, FLUCICLOVINE F-18, FLUCONAZOLE, FLUCYTOSINE, FLUDARABINE PHOSPHATE, FLUDEOXYGLUCOSE F-18, FLUDROCORTISONE ACETATE, FLUMAZENIL, FLUMETHASONE PIVALATE, FLUNISOLIDE, FLUOCINOLONE ACETONIDE, FLUOCINONIDE, FLUORESCEIN SODIUM, FLUORODOPA F-18, FLUOROESTRADIOL F-18, FLUOROMETHOLONE, FLUOROMETHOLONE ACETATE, FLUOROURACIL, FLUOXETINE HYDROCHLORIDE, FLUOXYMESTERONE, FLUPHENAZINE DECANOATE, FLUPHENAZINE ENANTHATE, FLUPHENAZINE HYDROCHLORIDE, FLUPREDNISOLONE, FLURANDRENOLIDE, FLURAZEPAM HYDROCHLORIDE, FLURBIPROFEN, FLURBIPROFEN SODIUM, FLUTAMIDE, FLUTEMETAMOL F-18, FLUTICASONE FUROATE, FLUTICASONE PROPIONATE, FLUVASTATIN SODIUM, FLUVOXAMINE MALEATE, FOLIC ACID, FOMEPIZOLE, FOMIVIRSEN SODIUM, FONDAPARINUX SODIUM, FORMOTEROL FUMARATE, FOSAMPRENAVIR CALCIUM, FOSAPREPITANT DIMEGLUMINE, FOSCARNET SODIUM, FOSDENOPTERIN HYDROBROMIDE, FOSFOMYCIN TROMETHAMINE, FOSINOPRIL SODIUM, FOSNETUPITANT CHLORIDE HYDROCHLORIDE, FOSPHENYTOIN SODIUM, FOSPROPOFOL DISODIUM, FOSTAMATINIB DISODIUM, FOSTEMSAVIR TROMETHAMINE, FROVATRIPTAN SUCCINATE, FULVESTRANT, FURAZOLIDONE, FUROSEMIDE, GABAPENTIN, GABAPENTIN ENACARBIL, GADOBENATE DIMEGLUMINE, GADOBUTROL, GADODIAMIDE, GADOFOSVESET TRISODIUM, GADOPENTETATE DIMEGLUMINE, GADOTERATE MEGLUMINE, GADOTERIDOL, GADOVERSETAMIDE, GADOXETATE DISODIUM, GALANTAMINE HYDROBROMIDE, GALLAMINE TRIETHIODIDE, GALLIUM CITRATE GA-67, GALLIUM DOTATATE GA-68, GALLIUM DOTATOC GA-68, GALLIUM GA-68 GOZETOTIDE, GALLIUM GA-68 PSMA-11, GALLIUM NITRATE, GANCICLOVIR, GANCICLOVIR SODIUM, GANIRELIX ACETATE, GATIFLOXACIN, GEFITINIB, GEMCITABINE HYDROCHLORIDE, GEMFIBROZIL, GEMIFLOXACIN MESYLATE, GENTAMICIN SULFATE, GENTIAN VIOLET, GILTERITINIB FUMARATE, GIVOSIRAN SODIUM, GLASDEGIB MALEATE, GLATIRAMER ACETATE, GLECAPREVIR, GLIMEPIRIDE, GLIPIZIDE, GLUCAGON, GLUCAGON HYDROCHLORIDE, GLUTETHIMIDE, GLYBURIDE, GLYCEROL PHENYLBUTYRATE, GLYCINE, GLYCOPYRROLATE, GLYCOPYRRONIUM TOSYLATE, GOLODIRSEN, GONADORELIN ACETATE, GONADORELIN HYDROCHLORIDE, GOSERELIN ACETATE, GRAMICIDIN, GRANISETRON, GRANISETRON HYDROCHLORIDE, GREPAFLOXACIN HYDROCHLORIDE, GRISEOFULVIN, GUAIFENESIN, GUANABENZ ACETATE, GUANADREL SULFATE, GUANETHIDINE MONOSULFATE, GUANFACINE HYDROCHLORIDE, GUANIDINE HYDROCHLORIDE, HALAZEPAM, HALCINONIDE, HALOBETASOL PROPIONATE, HALOFANTRINE HYDROCHLORIDE, HALOPERIDOL, HALOPERIDOL DECANOATE, HALOPERIDOL LACTATE, HALOPROGIN, HALOTHANE, HEPARIN CALCIUM, HEPARIN SODIUM, HETACILLIN, HETACILLIN POTASSIUM, HEXACHLOROPHENE, “HEXACHLOROPHENE, HEXAFLUORENIUM BROMIDE, HEXAMINOLEVULINATE HYDROCHLORIDE, HEXOCYCLIUM METHYLSULFATE, HEXYLCAINE HYDROCHLORIDE, HISTAMINE PHOSPHATE, HISTRELIN ACETATE, HOMATROPINE METHYLBROMIDE, HYDRALAZINE HYDROCHLORIDE, HYDROCHLOROTHIAZIDE, “HYDROCHLOROTHIAZIDE, HYDROCODONE BITARTRATE, HYDROCORTAMATE HYDROCHLORIDE, HYDROCORTISONE, HYDROCORTISONE ACETATE, HYDROCORTISONE BUTYRATE, HYDROCORTISONE CYPIONATE, HYDROCORTISONE PROBUTATE, HYDROCORTISONE SODIUM PHOSPHATE, HYDROCORTISONE SODIUM SUCCINATE, HYDROCORTISONE VALERATE, HYDROFLUMETHIAZIDE, HYDROGEN PEROXIDE, HYDROMORPHONE HYDROCHLORIDE, HYDROXOCOBALAMIN, HYDROXYAMPHETAMINE HYDROBROMIDE, HYDROXYCHLOROQUINE SULFATE, HYDROXYPROGESTERONE CAPROATE, HYDROXYPROPYL CELLULOSE, HYDROXYSTILBAMIDINE ISETHIONATE, HYDROXYUREA, HYDROXYZINE HYDROCHLORIDE, HYDROXYZINE PAMOATE, “HYDROXYZINE PAMOATE, IBANDRONATE SODIUM, IBREXAFUNGERP CITRATE, IBRUTINIB, IBUPROFEN, IBUPROFEN LYSINE, IBUPROFEN SODIUM, IBUTILIDE FUMARATE, ICATIBANT ACETATE, ICODEXTRIN, ICOSAPENT ETHYL, IDARUBICIN HYDROCHLORIDE, IDELALISIB, IDOXURIDINE, IFOSFAMIDE, ILOPERIDONE, ILOPROST, IMATINIB MESYLATE, IMIPRAMINE HYDROCHLORIDE, IMIPRAMINE PAMOATE, IMIQUIMOD, INAMRINONE LACTATE, INCLISIRAN SODIUM, INDACATEROL MALEATE, INDAPAMIDE, INDECAINIDE HYDROCHLORIDE, INDINAVIR SULFATE, INDIUM IN-111 CHLORIDE, INDIUM IN-111 OXYQUINOLINE, INDIUM IN-111 PENTETATE DIS ODIUM, INDIUM IN-111 PENTETREOTIDE KIT, INDOCYANINE GREEN, INDOMETHACIN, INDOMETHACIN SODIUM, INFIGRATINIB PHOSPHATE, INGENOL MEBUTATE, INOTERSEN SODIUM, INULIN, INVERT SUGAR, IOBENGUANE I-131, IOBENGUANE SULFATE I-123, IOBENGUANE SULFATE I-131, IOCETAMIC ACID, IODAMIDE MEGLUMINE, IODINE POVACRYLEX, IODIPAMIDE MEGLUMINE, IODIPAMIDE SODIUM, IODIXANOL, IODOHIPPURATE SODIUM I-123, IODOHIPPURATE SODIUM I-131, IODOXAMATE MEGLUMINE, IOFETAMINE HYDROCHLORIDE I-123, IOFLUPANE I-123, IOHEXOL, IOPAMIDOL, IOPANOIC ACID, IOPHENDYLATE, IOPROMIDE, IOTHALAMATE MEGLUMINE, IOTHALAMATE SODIUM, IOTHALAMATE SODIUM I-125, IOTROLAN, IOVERSOL, IOXAGLATE MEGLUMINE, IOXILAN, IPODATE CALCIUM, IPODATE SODIUM, IPRATROPIUM BROMIDE, IRBESARTAN, IRINOTECAN HYDROCHLORIDE, ISAVUCONAZONIUM SULFATE, ISOCARBOXAZID, ISOETHARINE HYDROCHLORIDE, ISOETHARINE MESYLATE, ISOFLURANE, ISOFLUROPHATE, ISONIAZID, ISOPROPAMIDE IODIDE, ISOPROPYL ALCOHOL, ISOPROTERENOL HYDROCHLORIDE, ISOPROTERENOL SULFATE, ISOSORBIDE, ISOSORBIDE DINITRATE, ISOSORBIDE MONONITRATE, ISOSULFAN BLUE, ISOTRETINOIN, ISRADIPINE, ISTRADEFYLLINE, ITRACONAZOLE, IVABRADINE, IVABRADINE HYDROCHLORIDE, IVACAFTOR, IVERMECTIN, IVOSIDENIB, IXABEPILONE, IXAZOMIB CITRATE, KANAMYCIN SULFATE, KETAMINE HYDROCHLORIDE, KETOCONAZOLE, KETOPROFEN, KETOROLAC TROMETHAMINE, KETOTIFEN FUMARATE, KRYPTON, L-GLUTAMINE, LABETALOL HYDROCHLORIDE, LACOSAMIDE, LACTITOL, LACTULOSE, LAMIVUDINE, LAMOTRIGINE, LANREOTIDE ACETATE, LANSOPRAZOLE, LANTHANUM CARBONATE, LAPATINIB DITOSYLATE, LAPYRIUM CHLORIDE, LAROTRECTINIB SULFATE, LASMIDITAN SUCCINATE, LATANOPROST, LATANOPROSTENE BUNOD, LEDIPASVIR, LEFAMULIN ACETATE, LEFLUNOMIDE, LEMBOREXANT, LENALIDOMIDE, LENVATINIB MESYLATE, LESINURAD, LETERMOVIR, LETROZOLE, LEUCOVORIN CALCIUM, LEUPROLIDE ACETATE, LEUPROLIDE MESYLATE, LEVALBUTEROL HYDROCHLORIDE, LEVALBUTEROL TARTRATE, LEVALLORPHAN TARTRATE, LEVAMISOLE HYDROCHLORIDE, LEVAMLODIPINE MALEATE, LEVETIRACETAM, LEVOBETAXOLOL HYDROCHLORIDE, LEVOBUNOLOL HYDROCHLORIDE, LEVOBUPIVACAINE HYDROCHLORIDE, LEVOCABASTINE HYDROCHLORIDE, LEVOCARNITINE, LEVOCETIRIZINE DIHYDROCHLORIDE, LEVODOPA, LEVOFLOXACIN, LEVOKETOCONAZOLE, LEVOLEUCOVORIN, LEVOLEUCOVORIN CALCIUM, LEVOMEPROMAZINE, LEVOMETHADYL ACETATE HYDROCHLORIDE, LEVOMILNACIPRAN HYDROCHLORIDE, LEVONORDEFRIN, LEVONORGESTREL, LEVOPROPDXYPHENE NAPSYLATE ANHYDROUS, LEVORPHANOL TARTRATE, LEVOTHYROXINE SODIUM, LIDOCAINE, LIDOCAINE HYDROCHLORIDE, LIFITEGRAST, LINACLOTIDE, LINAGLIPTIN, LINCOMYCIN, LINCOMYCIN HYDROCHLORIDE, LINDANE, LINEZOLID, LIOTHYRONINE SODIUM, LIOTRIX (T4, LIRAGLUTIDE RECOMBINANT, LISDEXAMFETAMINE DIMESYLATE, LISINOPRIL, LITHIUM CARBONATE, LITHIUM CITRATE, LODOXAMIDE TROMETHAMINE, LOFEXIDINE HYDROCHLORIDE, LOMEFLOXACIN HYDROCHLORIDE, LOMITAPIDE MESYLATE, LOMUSTINE, LONAFARNIB, LOPERAMIDE HYDROCHLORIDE, LOPINAVIR, LORACARBEF, LORATADINE, LORAZEPAM, LORLATINIB, LOSARTAN POTASSIUM, LOTEPREDNOL ETABONATE, LOVASTATIN, LOXAPINE, LOXAPINE HYDROCHLORIDE, LOXAPINE SUCCINATE, LUBIPROSTONE, LUCINACTANT, LULICONAZOLE, LUMASIRAN SODIUM, LUMATEPERONE TOSYLATE, LURASIDONE HYDROCHLORIDE, LURBINECTEDIN, LUSUTROMBOPAG, LUTETIUM DOTATATE LU-177, LUTETIUM LU-177 VIPIVOTIDE TETRAXETAN, LYPRESSIN, MACIMORELIN ACETATE, MACITENTAN, MAFENIDE ACETATE, MAGNESIUM ACETATE TETRAHYDRATE, MAGNESIUM CHLORIDE, MAGNESIUM HYDROXIDE, MAGNESIUM SULFATE, MALATHION, MANGAFODIPIR TRISODIUM, MANGANESE CHLORIDE, MANGANESE CHLORIDE TETRAHYDRATE, MANGANESE SULFATE, MANNITOL, MAPROTILINE HYDROCHLORIDE, MARALIXIBAT CHLORIDE, MARAVIROC, MARIBAVIR, MASOPROCOL, MAVACAMTEN, MAZINDOL, MEBENDAZOLE, MEBUTAMATE, MECAMYLAMINE HYDROCHLORIDE, MECHLORETHAMINE HYDROCHLORIDE, MECLIZINE HYDROCHLORIDE, MECLOCYCLINE SULFOSALICYLATE, MECLOFENAMATE SODIUM, MEDROXYPROGESTERONE ACETATE, MEDRYSONE, MEFENAMIC ACID, MEFLOQUINE HYDROCHLORIDE, MEGESTROL ACETATE, MELOXICAM, MELPHALAN, MELPHALAN FLUFENAMIDE HYDROCHLORIDE, MELPHALAN HYDROCHLORIDE, MEMANTINE HYDROCHLORIDE, MENADIOL SODIUM DIPHOSPHATE, MENADIONE, MENTHOL, MEPENZOLATE BROMIDE, MEPERIDINE HYDROCHLORIDE, MEPHENTERMINE SULFATE, MEPHENYTOIN, MEPIVACAINE HYDROCHLORIDE, MEPREDNISONE, MEPROBAMATE, MEQUINOL, MERCAPTOPURINE, MEROPENEM, MERSALYL SODIUM, MESALAMINE, MESNA, MESORIDAZINE BESYLATE, MESTRANOL, METAPROTERENOL SULFATE, METARAMINOL BITARTRATE, METAXALONE, METFORMIN HYDROCHLORIDE, METHACHOLINE CHLORIDE, METHACYCLINE HYDROCHLORIDE, METHADONE HYDROCHLORIDE, METHAMPHETAMINE HYDROCHLORIDE, METHANTHELINE BROMIDE, METHARBITAL, METHAZOLAMIDE, METHDILAZINE, METHDILAZINE HYDROCHLORIDE, METHENAMINE HIPPURATE, METHICILLIN SODIUM, METHIMAZOLE, METHIXENE HYDROCHLORIDE, METHOCARBAMOL, METHOHEXITAL SODIUM, METHOTREXATE, METHOTREXATE SODIUM, METHOXAMINE HYDROCHLORIDE, METHOXSALEN, METHSCOPOLAMINE BROMIDE, METHSUXIMIDE, METHYCLOTHIAZIDE, METHYL AMINOLEVULINATE HYDROCHLORIDE, METHYLDOPA, METHYLDOPATE HYDROCHLORIDE, METHYLENE BLUE, METHYLERGONOVINE MALEATE, METHYLNALTREXONE BROMIDE, METHYLPHENIDATE, METHYLPHENIDATE HYDROCHLORIDE, METHYLPREDNISOLONE, METHYLPREDNISOLONE ACETATE, METHYLPREDNISOLONE SODIUM SUCCINATE, METHYLTESTOSTERONE, METHYPRYLON, METHYSERGIDE MALEATE, METIPRANOLOL HYDROCHLORIDE, METOCLOPRAMIDE HYDROCHLORIDE, “METOCLOPRAMIDE HYDROCHLORIDE, METOCURINE IODIDE, METOLAZONE, METOPROLOL FUMARATE, METOPROLOL SUCCINATE, METOPROLOL TARTRATE, METRIZAMIDE, METRONIDAZOLE, METRONIDAZOLE HYDROCHLORIDE, METYRAPONE, METYROSINE, MEXILETINE HYDROCHLORIDE, MEZLOCILLIN SODIUM MONOHYDRATE, MICAFUNGIN SODIUM, MICONAZOLE, MICONAZOLE NITRATE, MIDAZOLAM, MIDAZOLAM HYDROCHLORIDE, MIDODRINE HYDROCHLORIDE, MIDOSTAURIN, MIFEPRISTONE, MIGALASTAT HYDROCHLORIDE, MIGLITOL, MIGLUSTAT, MILNACIPRAN HYDROCHLORIDE, MILRINONE LACTATE, MILTEFOSINE, MINOCYCLINE HYDROCHLORIDE, MINOXIDIL, MIPOMERSEN SODIUM, MIRABEGRON, MIRTAZAPINE, MISOPROSTOL, MITAPIVAT SULFATE, MITOMYCIN, MITOTANE, MITOXANTRONE HYDROCHLORIDE, MIVACURIUM CHLORIDE, MOB OCERTINIB SUCCINATE, MODAFINIL, MOEXIPRIL HYDROCHLORIDE, MOLINDONE HYDROCHLORIDE, MOMETASONE FUROATE, MONOBENZONE, MONOCTANOIN, MONOMETHYL FUMARATE, MONTELUKAST SODIUM, MORICIZINE HYDROCHLORIDE, MORPHINE SULFATE, MOXALACTAM DISODIUM, MOXIDECTIN, MOXIFLOXACIN HYDROCHLORIDE, MUPIROCIN, MUPIROCIN CALCIUM, MYCOPHENOLATE MOFETIL, MYCOPHENOLATE MOFETIL HYDROCHLORIDE, MYCOPHENOLIC ACID, MYCOPHENOLIC SODIUM, NABILONE, NABUMETONE, NADOLOL, NAFARELIN ACETATE, NAFCILLIN SODIUM, NAFTIFINE HYDROCHLORIDE, NALBUPHINE HYDROCHLORIDE, NALDEMEDINE TOSYLATE, NALIDIXIC ACID, NALMEFENE HYDROCHLORIDE, NALOXEGOL OXALATE, NALOXONE HYDROCHLORIDE, NALTREXONE, NALTREXONE HYDROCHLORIDE, NANDROLONE DECANOATE, NANDROLONE PHENPROPIONATE, NAPHAZOLINE HYDROCHLORIDE, NAPROXEN, NAPROXEN SODIUM, NARATRIPTAN HYDROCHLORIDE, NATAMYCIN, NATEGLINIDE, NEBIVOLOL HYDROCHLORIDE, NEDOCROMIL SODIUM, NEFAZODONE HYDROCHLORIDE, NELARABINE, NELFINAVIR MESYLATE, NEOMYCIN SULFATE, NEOSTIGMINE METHYLSULFATE, NEPAFENAC, NERATINIB MALEATE, NESIRITIDE RECOMBINANT, NETARSUDIL MESYLATE, NETILMICIN SULFATE, NETUPITANT, NEVIRAPINE, NIACIN, NIACINAMIDE, NICARDIPINE HYDROCHLORIDE, NICLOSAMIDE, NICOTINE, NICOTINE POLACRILEX, NIFEDIPINE, NIFURTIMOX, NILOTINIB HYDROCHLORIDE, NILUTAMIDE, NIMODIPINE, NINTEDANIB ESYLATE, NIRAPARIB TOSYLATE, NISOLDIPINE, NITAZOXANIDE, NITISINONE, NITRIC OXIDE, NITROFURANTOIN, NITROFURANTOIN SODIUM, NITROFURAZONE, NITROGLYCERIN, NIZATIDINE, NONOXYNOL-9, NOREPINEPHRINE BITARTRATE, NORETHINDRONE, NORETHINDRONE ACETATE, NORFLOXACIN, NORGESTREL, NORTRIPTYLINE HYDROCHLORIDE, NUSINERSEN SODIUM, NYSTATIN, OBETICHOLIC ACID, OCTREOTIDE ACETATE, ODEVIXIBAT, OFLOXACIN, OLANZAPINE, OLANZAPINE PAMOATE, OLAPARIB, OLICERIDINE, OLIVE OIL, OLMESARTAN MEDOXOMIL, OLODATEROL HYDROCHLORIDE, OLOPATADINE HYDROCHLORIDE, OLSALAZINE SODIUM, OMACETAXINE MEPESUCCINATE, OMADACYCLINE TOSYLATE, OMBITASVIR, OMEGA-3-ACID ETHYL ESTERS, OMEGA-3-ACID ETHYL ESTERS TYPE A, OMEGA-3-CARBOXYLIC ACIDS, OMEPRAZOLE, OMEPRAZOLE MAGNESIUM, ONDANSETRON, ONDANSETRON HYDROCHLORIDE, OPICAPONE, ORITAVANCIN DIPHOSPHATE, ORLISTAT, ORPHENADRINE CITRATE, ORPHENADRINE HYDROCHLORIDE, OSELTAMIVIR PHOSPHATE, OSILODROSTAT PHOSPHATE, OSIMERTINIB MESYLATE, OSPEMIFENE, OTESECONAZOLE, OXACILLIN SODIUM, OXALIPLATIN, OXAMNIQUINE, OXANDROLONE, OXAPROZIN, OXAPROZIN POTASSIUM, OXAZEPAM, OXCARBAZEPINE, OXICONAZOLE NITRATE, OXPRENOLOL HYDROCHLORIDE, OXTRIPHYLLINE, OXYBUTYNIN, OXYBUTYNIN CHLORIDE, OXYCODONE, OXYCODONE HYDROCHLORIDE, OXYMETAZOLINE HYDROCHLORIDE, OXYMETHOLONE, OXYMORPHONE HYDROCHLORIDE, OXYPHENBUTAZONE, OXYPHENCYCLIMINE HYDROCHLORIDE, OXYPHENONIUM BROMIDE, OXYTETRACYCLINE, OXYTETRACYCLINE CALCIUM, OXYTETRACYCLINE HYDROCHLORIDE, OXYTOCIN, OZANIMOD HYDROCHLORIDE, OZENOXACIN, PACLITAXEL, PACRITINIB CITRATE, PAFOLACIANINE SODIUM, PALBOCICLIB, PALIPERIDONE, PALIPERIDONE PALMITATE, PALONOSETRON HYDROCHLORIDE, PAMIDRONATE DISODIUM, PANCURONIUM BROMIDE, PANOBINOSTAT LACTATE, PANTOPRAZOLE SODIUM, PARAMETHADIONE, PARAMETHASONE ACETATE, PARGYLINE HYDROCHLORIDE, PARICALCITOL, PAROMOMYCIN SULFATE, PAROXETINE HYDROCHLORIDE, PAROXETINE MESYLATE, PASIREOTIDE DIASPARTATE, PASIREOTIDE PAMOATE, PATIROMER SORBITEX CALCIUM, PATISIRAN SODIUM, PAZOPANIB HYDROCHLORIDE, PEGAPTANIB SODIUM, PEGCETACOPLAN, PEGINESATIDE ACETATE, PEMETREXED, PEMETREXED DISODIUM, PEMIGATINIB, PEMIROLAST POTASSIUM, PEMOLINE, PENBUTOLOL SULFATE, PENCICLOVIR, PENICILLAMINE, PENICILLIN G BENZATHINE, PENICILLIN G POTASSIUM, PENICILLIN G PROCAINE, PENICILLIN G SODIUM, PENICILLIN V, PENICILLIN V POTASSIUM, PENTAGASTRIN, PENTAMIDINE ISETHIONATE, PENTAZOCINE HYDROCHLORIDE, PENTAZOCINE LACTATE, PENTETATE CALCIUM TRISODIUM, PENTETATE CALCIUM TRISODIUM YB-169, PENTETATE ZINC TRISODIUM, PENTOBARBITAL, PENTOBARBITAL SODIUM, PENTOLINIUM TARTRATE, PENTOSAN POLYSULFATE SODIUM, PENTOSTATIN, PENTOXIFYLLINE, PERAMIVIR, PERAMPANEL, PERFLUBRON, PERFLUOROPOLYMETHYLISOPROPYL ETHER, PERFLUTREN, PERGOLIDE MESYLATE, PERINDOPRIL ERBUMINE, PERMETHRIN, PERPHENAZINE, PEXIDARTINIB HYDROCHLORIDE, PHENACEMIDE, PHENAZOPYRIDINE HYDROCHLORIDE, PHENDIMETRAZINE TARTRATE, PHENELZINE SULFATE, PHENINDIONE, PHENMETRAZINE HYDROCHLORIDE, PHENOXYBENZAMINE HYDROCHLORIDE, PHENPROCOUMON, PHENSUXIMIDE, PHENTERMINE HYDROCHLORIDE, PHENTERMINE RESIN COMPLEX, PHENTOLAMINE MESYLATE, PHENYL AMINOSALICYLATE, PHENYLBUTAZONE, PHENYLEPHRINE HYDROCHLORIDE, PHENYTOIN, PHENYTOIN SODIUM, PHYTONADIONE, PIFLUFOLASTAT F-18, PILOCARPINE, PILOCARPINE HYDROCHLORIDE, PIMAVANSERIN TARTRATE, PIMECROLIMUS, PIMOZIDE, PINACIDIL, PINDOLOL, PIOGLITAZONE HYDROCHLORIDE, PIPECURONIUM BROMIDE, PIPERACETAZINE, PIPERACILLIN SODIUM, PIPERAZINE CITRATE, PIPERONYL BUTOXIDE, PIPOBROMAN, PIRBUTEROL ACETATE, PIRFENIDONE, PIROXICAM, PITAVASTATIN CALCIUM, PITAVASTATIN MAGNESIUM, PITAVASTATIN SODIUM, PITOLISANT HYDROCHLORIDE, PLAZOMICIN SULFATE, PLECANATIDE, PLERIXAFOR, PLICAMYCIN, PODOFILOX, POLIDOCANOL, POLYESTRADIOL PHOSPHATE, POLYETHYLENE GLYCOL 3350, POLYMYXIN B SULFATE, POLYTHIAZIDE, POMALIDOMIDE, PONATINIB HYDROCHLORIDE, PONESIMOD, PORFIMER SODIUM, POSACONAZOLE, POTASSIUM ACETATE, POTASSIUM AMINOSALICYLATE, POTASSIUM CHLORIDE, POTASSIUM CITRATE, POTASSIUM IODIDE, POTASSIUM PERCHLORATE, POTASSIUM PHOSPHATE, POVIDONE-IODINE, PRALATREXATE, PRALIDOXIME CHLORIDE, PRALSETINIB, PRAMIPEXOLE DIHYDROCHLORIDE, PRAMLINTIDE ACETATE, PRASTERONE, PRASUGREL HYDROCHLORIDE, PRAVASTATIN SODIUM, PRAZEPAM, PRAZIQUANTEL, PRAZOSIN HYDROCHLORIDE, PREDNICARBATE, PREDNISOLONE, PREDNISOLONE ACETATE, PREDNISOLONE SODIUM PHOSPHATE, PREDNISOLONE TEBUTATE, PREDNISONE, PREGABALIN, PRETOMANID, PRILOCAINE HYDROCHLORIDE, PRIMAQUINE PHOSPHATE, PRIMIDONE, PROBENECID, PROBUCOL, PROCAINAMIDE HYDROCHLORIDE, PROCAINE HYDROCHLORIDE, PROCAINE MERETHOXYLLINE, PROCARBAZINE HYDROCHLORIDE, PROCHLORPERAZINE, PROCHLORPERAZINE EDISYLATE, PROCHLORPERAZINE MALEATE, PROCYCLIDINE HYDROCHLORIDE, PROGESTERONE, PROMAZINE HYDROCHLORIDE, PROMETHAZINE HYDROCHLORIDE, PROPAFENONE HYDROCHLORIDE, PROPANTHELINE BROMIDE, PROPARACAINE HYDROCHLORIDE, PROPIOLACTONE, PROPIOMAZINE HYDROCHLORIDE, PROPOFOL, PROPDXYPHENE HYDROCHLORIDE, PROPDXYPHENE NAPSYLATE, PROPRANOLOL HYDROCHLORIDE, PROPYLIODONE, PROPYLTHIOURACIL, PROTAMINE SULFATE, PROTEIN HYDROLYSATE, PROTIRELIN, PROTOKYLOL HYDROCHLORIDE, PROTRIPTYLINE HYDROCHLORIDE, PRUCALOPRIDE SUCCINATE, PSEUDOEPHEDRINE HYDROCHLORIDE, PSEUDOEPHEDRINE POLISTIREX, PSEUDOEPHEDRINE SULFATE, PURIFIED WATER, PYRAZINAMIDE, PYRIDOSTIGMINE BROMIDE, PYRIDOXINE HYDROCHLORIDE, PYRILAMINE MALEATE, PYRIMETHAMINE, PYRITHIONE ZINC, PYRVINIUM PAMOATE, QUAZEPAM, QUETIAPINE FUMARATE, QUINAPRIL HYDROCHLORIDE, QUINESTROL, QUINETHAZONE, QUINIDINE GLUCONATE, QUINIDINE POLYGALACTURONATE, QUINIDINE SULFATE, QUININE SULFATE, RABEPRAZOLE SODIUM, RADIUM RA-223 DICHLORIDE, RALOXIFENE HYDROCHLORIDE, RALTEGRAVIR POTASSIUM, RAMELTEON, RAMIPRIL, RANITIDINE BISMUTH CITRATE, RANITIDINE HYDROCHLORIDE, RANOLAZINE, RAPACURONIUM BROMIDE, RASAGILINE MESYLATE, RAUWOLFIA SERPENTINA ROOT, REGADENOSON, REGORAFENIB, RELUGOLIX, REMDESIVIR, REMIFENTANIL HYDROCHLORIDE, REMIMAZOLAM BESYLATE, REPAGLINIDE, RESCINNAMINE, RESERPINE, RETAPAMULIN, REVEFENACIN, RIBAVIRIN, RIBOCICLIB SUCCINATE, RIBOFLAVIN 5′-PHOSPHATE SODIUM, RIFABUTIN, RIFAMPIN, RIFAMYCIN SODIUM, RIFAPENTINE, RIFAXIMIN, RILPIVIRINE HYDROCHLORIDE, RILUZOLE, RIMANTADINE HYDROCHLORIDE, RIMEGEPANT SULFATE, RIMEXOLONE, RIOCIGUAT, RIPRETINIB, RISDIPLAM, RISEDRONATE SODIUM, RISPERIDONE, RITODRINE HYDROCHLORIDE, RITONAVIR, RIVAROXABAN, RIVASTIGMINE, RIVASTIGMINE TARTRATE, RIZATRIPTAN BENZOATE, ROCURONIUM BROMIDE, ROFECOXIB, ROFLUMILAST, ROLAPITANT HYDROCHLORIDE, ROMIDEPSIN, ROPINIROLE HYDROCHLORIDE, ROPIVACAINE HYDROCHLORIDE, ROSE BENGAL SODIUM I-131, ROSIGLITAZONE MALEATE, ROSUVASTATIN CALCIUM, ROTIGOTINE, RUBIDIUM CHLORIDE RB-82, RUCAPARIB CAMSYLATE, RUFINAMIDE, RUXOLITINIB PHOSPHATE, SACUBITRIL, SAFFLOWER OIL, SAFINAMIDE MESYLATE, SALMETEROL XINAFOATE, SAMARIUM SM-153 LEXIDRONAM PENTASODIUM, SAPROPTERIN DIHYDROCHLORIDE, SAQUINAVIR, SAQUINAVIR MESYLATE, SARALASIN ACETATE, SARECYCLINE HYDROCHLORIDE, SAXAGLIPTIN HYDROCHLORIDE, SCOPOLAMINE, SECNIDAZOLE, SECOBARBITAL SODIUM, SECRETIN, SECRETIN SYNTHETIC HUMAN, SECRETIN SYNTHETIC PORCINE, SELEGILINE, SELEGILINE HYDROCHLORIDE, SELENIOUS ACID, SELENIUM SULFIDE, SELENOMETHIONINE SE-75, SELEXIPAG, SELINEXOR, SELPERCATINIB, SELUMETINIB SULFATE, SEMAGLUTIDE, SERMORELIN ACETATE, SERTACONAZOLE NITRATE, SERTRALINE HYDROCHLORIDE, SETMELANOTIDE ACETATE, SEVELAMER CARBONATE, SEVELAMER HYDROCHLORIDE, SEVOFLURANE, SIBUTRAMINE HYDROCHLORIDE, SILDENAFIL CITRATE, SILODOSIN, SILVER SULFADIAZINE, SIMEPREVIR SODIUM, SIMETHICONE-CELLULOSE, SIMVASTATIN, SINCALIDE, SINECATECHINS, SIPONIMOD FUMARIC ACID, SIROLIMUS, SITAGLIPTIN PHOSPHATE, SODIUM ACETATE, SODIUM BENZOATE, SODIUM BICARBONATE, SODIUM CHLORIDE, SODIUM CHROMATE CR-51, SODIUM FLUORIDE F-18, SODIUM FLUORIDE, SODIUM IODIDE I-123, SODIUM IODIDE I-131, SODIUM LACTATE, SODIUM MONOFLUOROPHOSPHATE, SODIUM NITRITE, SODIUM NITROPRUSSIDE, SODIUM OXYBATE, SODIUM PHENYLBUTYRATE, SODIUM PHOSPHATE, SODIUM PHOSPHATE P-32, SODIUM POLYSTYRENE SULFONATE, SODIUM SUCCINATE, SODIUM TETRADECYL SULFATE, SODIUM THIOSULFATE, SODIUM ZIRCONIUM CYCLOSILICATE, SOFOSBUVIR, SOLIFENACIN SUCCINATE, SOLRIAMFETOL HYDROCHLORIDE, SONIDEGIB PHOSPHATE, SORAFENIB TOSYLATE, SORBITOL, SOTALOL HYDROCHLORIDE, SOTORASIB, SOYBEAN OIL, SPARFLOXACIN, SPECTINOMYCIN HYDROCHLORIDE, SPINOSAD, SPIRAPRIL HYDROCHLORIDE, SPIRONOLACTONE, STANOZOLOL, STAVUDINE, STERILE WATER FOR INJECTION, STERILE WATER FOR IRRIGATION, STIRIPENTOL, STREPTOMYCIN SULFATE, STREPTOZOCIN, STRONTIUM CHLORIDE SR-89, SUCCIMER, SUCCINYLCHOLINE CHLORIDE, SUCRALFATE, SUFENTANIL CITRATE, SUGAMMADEX SODIUM, SULCONAZOLE NITRATE, SULFACETAMIDE SODIUM, SULFACYTINE, SULFADIAZINE, SULFADIAZINE SODIUM, SULFAMETER, SULFAMETHIZOLE, SULFAMETHOXAZOLE, SULFANILAMIDE, SULFAPHENAZOLE, SULFAPYRIDINE, SULFASALAZINE, SULFINPYRAZONE, SULFISOXAZOLE, SULFISOXAZOLE ACETYL, SULFISOXAZOLE DIOLAMINE, SULFOXONE SODIUM, SULFUR, SULFUR HEXAFLUORIDE LIPID-TYPE A MICROSPHERES, SULINDAC, SUMATRIPTAN, SUMATRIPTAN SUCCINATE, SUNITINIB MALATE, SUPROFEN, SUVOREXANT, TACRINE HYDROCHLORIDE, TACROLIMUS, TADALAFIL, TAFAMIDIS, TAFAMIDIS MEGLUMINE, TAFENOQUINE SUCCINATE, TAFLUPROST, TALAZOPARIB TOSYLATE, TALBUTAL, TALC, TAMOXIFEN CITRATE, TAMSULOSIN HYDROCHLORIDE, TAPENTADOL HYDROCHLORIDE, TASIMELTEON, TAVABOROLE, TAZAROTENE, TAZEMETOSTAT HYDROBROMIDE, TECHNETIUM TC-99M APCITIDE, TECHNETIUM TC-99M BICISATE KIT, TECHNETIUM TC-99M DEPREOTIDE, TECHNETIUM TC-99M DISOFENIN KIT, TECHNETIUM TC-99M ETIDRONATE KIT, TECHNETIUM TC-99M EXAMETAZIME KIT, TECHNETIUM TC-99M FERPENTETATE KIT, TECHNETIUM TC-99M GLUCEPTATE KIT, TECHNETIUM TC-99M LIDOFENIN KIT, TECHNETIUM TC-99M MEBROFENIN KIT, TECHNETIUM TC-99M MEDRONATE, TECHNETIUM TC-99M MEDRONATE KIT, TECHNETIUM TC-99M MERTIATIDE KIT, TECHNETIUM TC-99M OXIDRONATE KIT, TECHNETIUM TC-99M PENTETATE KIT, TECHNETIUM TC-99M POLYPHOSPHATE KIT, TECHNETIUM TC-99M PYRO/TRIMETA PHOSPHATES KIT, TECHNETIUM TC-99M PYROPHOSPHATE KIT, TECHNETIUM TC-99M RED BLOOD CELL KIT, TECHNETIUM TC-99M SESTAMIBI KIT, TECHNETIUM TC-99M SODIUM PERTECHNETATE, TECHNETIUM TC-99M SODIUM PERTECHNETATE GENERATOR, TECHNETIUM TC-99M SUCCIMER, TECHNETIUM TC-99M SUCCIMER KIT, TECHNETIUM TC-99M SULFUR COLLOID, TECHNETIUM TC-99M SULFUR COLLOID KIT, TECHNETIUM TC-99M TEBOROXIME KIT, TECHNETIUM TC-99M TETROFOSMIN KIT, TECHNETIUM TC-99M TILMANOCEPT, TECOVIRIMAT, TEDIZOLID PHOSPHATE, TEDUGLUTIDE RECOMBINANT, TEGASEROD MALEATE, TELAPREVIR, TELAVANCIN HYDROCHLORIDE, TELBIVUDINE, TELITHROMYCIN, TELMISARTAN, TELOTRISTAT ETIPRATE, TEMAZEPAM, TEMOZOLOMIDE, TEMSIROLIMUS, TENAPANOR HYDROCHLORIDE, TENIPOSIDE, TENOFOVIR ALAFENAMIDE FUMARATE, TENOFOVIR DISOPROXIL FUMARATE, TEPOTINIB HYDROCHLORIDE, TERAZOSIN HYDROCHLORIDE, TERBINAFINE, TERBINAFINE HYDROCHLORIDE, TERBUTALINE SULFATE, TERCONAZOLE, TERIFLUNOMIDE, TERIPARATIDE, TERIPARATIDE ACETATE, TESTOLACTONE, TESTOSTERONE, TESTOSTERONE CYPIONATE, TESTOSTERONE ENANTHATE, TESTOSTERONE PROPIONATE, TESTOSTERONE UNDECANOATE, TETRABENAZINE, TETRACAINE HYDROCHLORIDE, TETRACYCLINE HYDROCHLORIDE, TETRACYCLINE PHOSPHATE COMPLEX, TETRAHYDROZOLINE HYDROCHLORIDE, THALIDOMIDE, THALLOUS CHLORIDE TL-201, THEOPHYLLINE, THEOPHYLLINE SODIUM GLYCINATE, THIABENDAZOLE, THIAMINE HYDROCHLORIDE, THIAMYLAL SODIUM, THIETHYLPERAZINE MALATE, THIETHYLPERAZINE MALEATE, THIOGUANINE, THIOPENTAL SODIUM, THIORIDAZINE, THIORIDAZINE HYDROCHLORIDE, THIOTEPA, THIOTHIXENE, THIOTHIXENE HYDROCHLORIDE, TIAGABINE HYDROCHLORIDE, TICAGRELOR, TICARCILLIN DISODIUM, TICLOPIDINE HYDROCHLORIDE, TIGECYCLINE, TILUDRONATE DISODIUM, TIMOLOL, TIMOLOL MALEATE, TINIDAZOLE, TINZAPARIN SODIUM, TIOCONAZOLE, TIOPRONIN, TIOTROPIUM BROMIDE, TIPIRACIL HYDROCHLORIDE, TIPRANAVIR, TIRBANIBULIN, TIROFIBAN HYDROCHLORIDE, TIVOZANIB HYDROCHLORIDE, TIZANIDINE HYDROCHLORIDE, TOBRAMYCIN, TOBRAMYCIN SULFATE, TOCAINIDE HYDROCHLORIDE, TOFACITINIB CITRATE, TOLAZAMIDE, TOLAZOLINE HYDROCHLORIDE, TOLBUTAMIDE, TOLBUTAMIDE SODIUM, TOLCAPONE, TOLMETIN SODIUM, TOLTERODINE TARTRATE, TOLVAPTAN, TOPIRAMATE, TOPOTECAN HYDROCHLORIDE, TOREMIFENE CITRATE, TORSEMIDE, TRABECTEDIN, TRAMADOL HYDROCHLORIDE, TRAMETINIB DIMETHYL SULFOXIDE, TRANDOLAPRIL, TRANEXAMIC ACID, TRANYLCYPROMINE SULFATE, TRAVOPROST, TRAZODONE HYDROCHLORIDE, TREPROSTINIL, TREPROSTINIL DIOLAMINE, TRETINOIN, TRIAMCINOLONE, TRIAMCINOLONE ACETONIDE, TRIAMCINOLONE DIACETATE, TRIAMCINOLONE HEXACETONIDE, TRIAMTERENE, TRIAZOLAM, TRICHLORMETHIAZIDE, TRICLABENDAZOLE, TRICLOFOS SODIUM, TRIDIHEXETHYL CHLORIDE, TRIENTINE HYDROCHLORIDE, TRIENTINE TETRAHYDROCHLORIDE, TRIFAROTENE, TRIFLUOPERAZINE HYDROCHLORIDE, TRIFLUPROMAZINE, TRIFLUPROMAZINE HYDROCHLORIDE, TRIFLURIDINE, TRIHEPTANOIN, TRIHEXYPHENIDYL HYDROCHLORIDE, TRILACICLIB DIHYDROCHLORIDE, TRILOSTANE, TRIMEPRAZINE TARTRATE, TRIMETHADIONE, TRIMETHAPHAN CAMSYLATE, TRIMETHOBENZAMIDE HYDROCHLORIDE, TRIMETHOPRIM, TRIMETHOPRIM HYDROCHLORIDE, TRIMETREXATE GLUCURONATE, TRIMIPRAMINE MALEATE, TRIOXSALEN, TRIPELENNAMINE CITRATE, TRIPELENNAMINE HYDROCHLORIDE, TRIPLE SULFA (SULFABENZAMIDE, TRIPROLIDINE HYDROCHLORIDE, TRIPTORELIN PAMOATE, TRISULFAPYRIMIDINES (SULFADIAZINE, TROGLITAZONE, TROLAMINE POLYPEPTIDE OLEATE CONDENSATE, TROLEANDOMYCIN, TROMETHAMINE, TROPICAMIDE, TROSPIUM CHLORIDE, TROVAFLOXACIN MESYLATE, TRYPAN BLUE, TUBOCURARINE CHLORIDE, TUCATINIB, TYROPANOATE SODIUM, UBROGEPANT, ULIPRISTAL ACETATE, UMBRALISIB TOSYLATE, UMECLIDINIUM BROMIDE, UNOPROSTONE ISOPROPYL, UPADACITINIB, URACIL MUSTARD, UREA, UREA C-13, URIDINE TRIACETATE, URSODIOL, VALACYCLOVIR HYDROCHLORIDE, VALBENAZINE TOSYLATE, VALDECOXIB, VALGANCICLOVIR HYDROCHLORIDE, VALPROATE SODIUM, VALPROIC ACID, VALRUBICIN, VALSARTAN, VANCOMYCIN, VANCOMYCIN HYDROCHLORIDE, VANDETANIB, VARDENAFIL HYDROCHLORIDE, VARENICLINE TARTRATE, VASOPRESSIN, VASOPRESSIN TANNATE, VECURONIUM BROMIDE, VEMURAFENIB, VENETOCLAX, VENLAFAXINE HYDROCHLORIDE, VERAPAMIL HYDROCHLORIDE, VERATRUM VIRIDE ROOT, VERICIGUAT, VERTEPORFIN, VIBEGRON, VIDARABINE, VIGABATRIN, VILAZODONE HYDROCHLORIDE, VILOXAZINE HYDROCHLORIDE, VILTOLARSEN, VINBLASTINE SULFATE, VINCRISTINE SULFATE, VINORELBINE TARTRATE, VIOMYCIN SULFATE, VISMODEGIB, VITAMIN A, VITAMIN A PALMITATE, VOCLOSPORIN, VORAPAXAR SULFATE, VORICONAZOLE, VORINOSTAT, VORTIOXETINE HYDROBROMIDE, VOSORITIDE, VOXELOTOR, WARFARIN POTASSIUM, WARFARIN SODIUM, XENON XE-127, XENON XE-133, XYLOSE, ZAFIRLUKAST, ZALCITABINE, ZALEPLON, ZANAMIVIR, ZANUBRUTINIB, ZICONOTIDE ACETATE, ZIDOVUDINE, ZILEUTON, ZINC ACETATE, ZINC CHLORIDE, ZINC SULFATE, ZIPRASIDONE HYDROCHLORIDE, ZIPRASIDONE MESYLATE, ZOLEDRONIC ACID, ZOLMITRIPTAN, ZOLPIDEM TARTRATE, ZONISAMIDE,

Embodiments of the present invention are directed to identify new usage of known drugs and compounds, drug repurposing, as potential nuclear receptor modulators using a nuclear receptor cofactor recruitment-based profiling system and using a drug repurposing computing device. With the drug repurposing computing device and a nuclear receptor cofactor recruitment screening assay of the present invention, new uses other than the original medical indication of the known drug can be identified. Finding new indications for such drugs will benefit patients who require a potential new therapy targeting said nuclear receptor sooner since known drugs usually have an acceptable safety and pharmacokinetic profile.

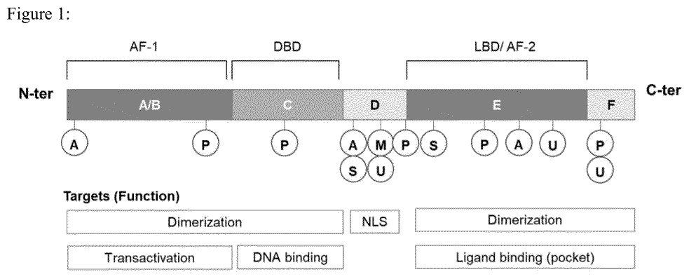

The nuclear receptor of the nuclear receptor cofactor recruitment-based profiling system using a drug repurposing computing device can be a truncated protein such as the ligand-binding domain of a nuclear receptor. The ligand-binding domain contains a hydrophobic-binding pocket that attracts the ligand or in the case of the invention the repurposing drug. The binding of the ligand or repurposing drug alters the conformation of the ligand-binding domain, resulting in the ligand or repurposing drug being trapped within the hydrophobic environment. Functions attributed to the ligand-binding domain include ligand or repurposing drug binding, heat shock protein-90 binding, transcriptional activation, and dimerization. Heat shock proteins are released from the ligand-binding domain following ligand or repurposing drug binding allowing exposure of the dimerization motif, a regulatory zipper domain of the ligand-binding domain. The activation function-2 domain, a highly conserved domain within the nuclear receptor superfamily, is found in the C-terminal end of the ligand-binding domain and is recognized by various transcriptional co-regulatory proteins which are used in the nuclear receptor cofactor recruitment-based profiling system. In contrast to the ligand-binding domain of the nuclear receptor a full-length nuclear receptor that contains all parts of the nuclear receptor as outlined in FIG. 1 can also be used.

One method to identify the effect of ligands or drug molecules such as glucocorticoid receptor modulators on the nuclear receptor-coregulator interactions is using microarrays^{xxv}. Such a microarray technology described in the art consists of several of many unique coregulator-derived peptides spotted onto the microarray.

In certain embodiments of the invention, a probability or similarity score is used to determine the likelihood of a repurposed drug acting as a nuclear receptor modulator using the nuclear receptor cofactor recruitment screening assay. This probability score can be determined using a class discovery method which is sometimes called unsupervised classification where one tries to identify whether the peptides used in the cofactor recruitment assay cluster together in groups. Identifying naturally existing groups of cofactor peptides that cluster together can enable the discovery of new groups that otherwise were not previously known to exist. Cofactor recruitment data obtained from a set of cofactor peptides is used to identify novel clusters or classes of cofactors. This type of approach is not hypothesis-driven but rather is based on iterative pattern recognition or statistical learning methods to find an “optimal” number of clusters in the cofactor data. Examples of unsupervised analysis methods include self-organizing maps, neural gas, k-means cluster analyses, hierarchical cluster analysis, Genomic Signal Processing based clustering, and model-based cluster analysis. For some of these methods, a person skilled in the art can measure the distance between groups of cofactor peptides. The class discovery analyses can be used to predict which nuclear receptor drugs have a favorable cofactor binding profile and which nuclear receptor drugs which for instance have failed during late-stage clinical have unfavorable cofactor binding profiles. These cofactor binding profiles can be used in a process called class prediction analysis. This approach, also called supervised classification, is used to develop a predictive model which is based on known drugs and used to predict if unknown repurposed drugs have a favorable cofactor binding profile or unfavorable cofactor binding profile, or no cofactor binding profile. Said supervised analysis for class prediction involves the use of techniques such as linear regression, k-nearest neighbor, learning vector quantization, decision tree analysis, random forests, naive Bayes, logistic regression, kernel regression, artificial neural networks, and support vector machines. Using these class discovery or class prediction algorithms can be used to generate a probability score for a repurposed drug to have certain favorable nuclear receptor modulation properties.

The cofactors of the nuclear receptor cofactor recruitment-based profiling system using a drug repurposing computing device can be an assay consisting of several truncated cofactor proteins or peptides. Such peptides are derived from the protein sequence of said cofactor proteins and have a sequence of a nuclear receptor binding motif, including the LXXLL or LXXXIXXXL sequence, whereby the L is a leucine amino acid and an I is an isoleucine amino acid, and an X is any amino acid sequence. The peptides usually have a length ranging between 10 and 40 amino acids, preferably a length of 20 to 30 amino acids and a typical peptide length of 25 amino acids. Within the methods of the present invention the number of cofactor peptides used in the nuclear receptor cofactor recruitment-based profiling system ranges between 2 and 1000 cofactor peptides. A very suitable number of cofactor peptides ranges from 2-50 and a typical number of cofactor peptides used within the methods of the present invention is at least 2-30.

In certain embodiments, the said nuclear receptor used in the nuclear receptor cofactor recruitment screening assay is a full-length nuclear receptor derived from a cell lysate. Cell lysates are generated through cell lysis which is the process of breaking open cells and purifying or further studying their contents such as nuclear receptors. Lysis may be affected by enzymes or detergents or other chaotropic agents. Mechanical lysis or disruption of cell membranes, as by repeated freezing and thawing, sonication, pressure, and for instance bead or bead-mill systems are known techniques in the art for disruption of cell membranes.

The nuclear receptor proteins used in the nuclear receptor cofactor recruitment-based profiling system are selected from the list of NR0A1, NR0B1, NR0B2, NR1A1, NR1A2, NR1B1, NR1B2, NR1B3, NR1C1, NR1C2, NR1C3, NR1D1, NR1D2, NR1E1, NR1F1, NR1F2, NR1F3, NR1G1, NR1H1, NR1H2, NR1H3, NR1H4, NR1H5, NR1I1, NR1I2, NR1I3, NR1J1, NR1J2, NR1J3, NR1K1, NR2A1, NR2A2, NR2B1, NR2B2, NR2B3, NR2B4, NR2C1, NR2C2, NR2E1, NR2E3, NR2F1, NR2F2, NR2F6, NR3A1, NR3A2, NR3B1, NR3B2, NR3B3, NR3C1, NR3C2, NR3C3, NR3C4, NR3D, NR3E, NR3F, NR4A1, NR4A2, NR4A3, NR5A1, NR5A2, NR5B1, NR6A1, NR7A1, NR7B1, NR7C1, and NR8A1.

In certain embodiments, nuclear receptors described herein comprise wild-type nuclear receptor variant and one or more mutant nuclear receptor variants. The identification of naturally occurring nuclear receptor mutations over the last 30 years has provided insights into their structure and function although there are still many nuclear receptors for which an associated disorder has not yet been discovered. More recently, whole-exome sequencing has associated pathogenic genetic variants with unexpected, often multisystem, human phenotypes. To date, defects in 20 of 48 human NR genes have been associated with human disorders, with different mutations mediating phenotypes of varying severity or several distinct conditions being associated with different changes in the same gene. Studies of individuals with deleterious genetic variants can elucidate novel roles of human nuclear receptors, validating them as targets for drug development or providing new insights into structure-function relationships. Importantly, human genetic discoveries enable definitive disease diagnosis and can provide opportunities to therapeutically manage affected individuals^{xxvi}.

In yet another embodiment the methods or uses as taught herein describe wild-type nuclear receptor variant and one or more nuclear receptor variants each containing one or more post-translational modifications such as acetylation, methylation, phosphorylation, SUMOylation, and ubiquitination.

### Example 1

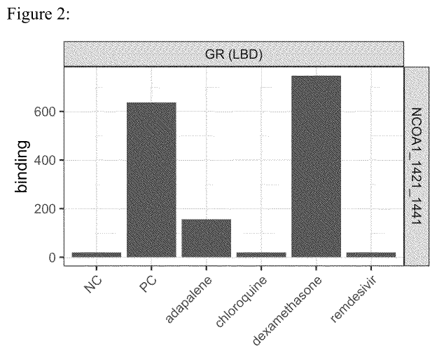

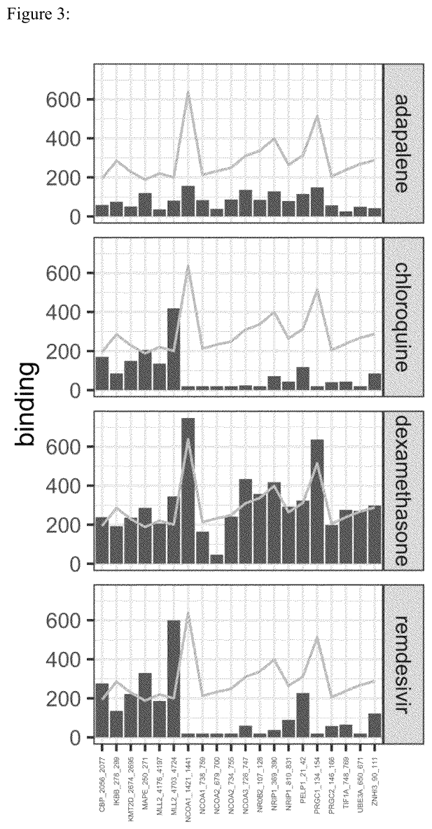

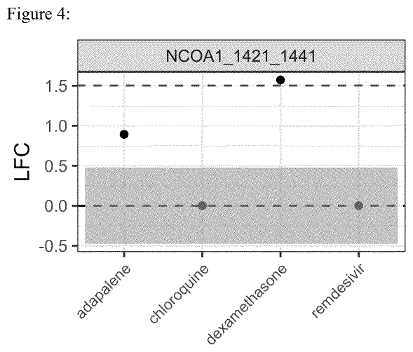

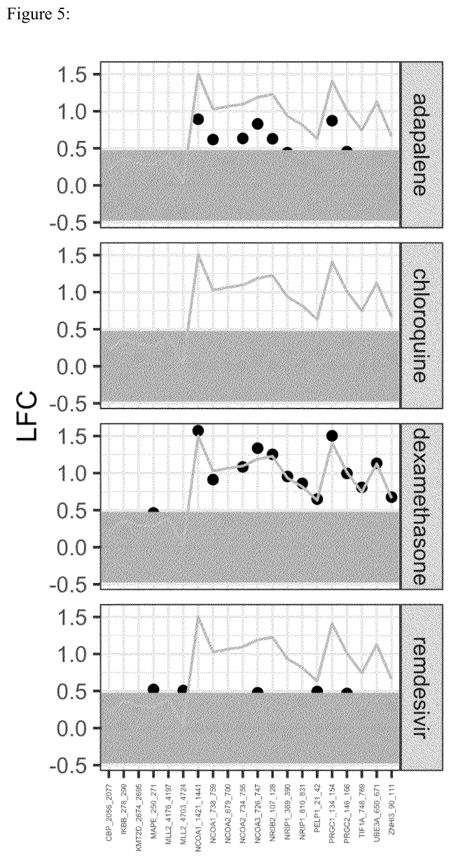

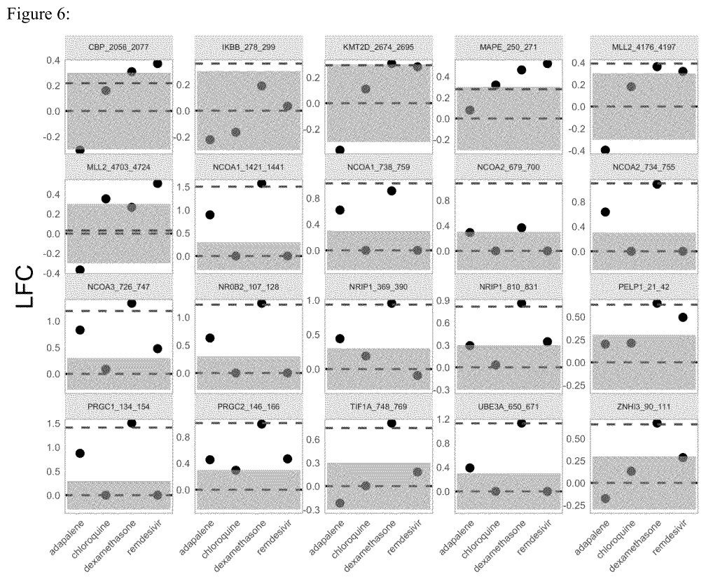

Interactions between the glucocorticoid receptor ligand binding domain and the selected drugs for repurposing and coregulator peptides were determined using the method as described by Zalachoras et al 2013^{xxvii} Each peptide array consisting of 70 coregulator peptides as described in table 1 were incubated with a reaction mixture of 1 nM purified glucocorticoid receptor recombinant human protein, ligand binding domain (LBD), (Thermo Fisher Scientific, cat #A15668), ALEXA488-conjugated anti-GST antibody and buffer F (PV4689, A-11131, and PV4547; Invitrogen). The drugs selected for repositioning were: adapalene, dexamethasone, remdesivir, chloroquine, vs the positive control, cortisol (table 5). For ligand induced cofactor peptide interaction profiling the set of repurposing drugs were tested at a concentration of 0.05 mM compared to solvent (2% DMSO in water). Incubation was performed at 20° C. on the cofactor peptide array. Glucocorticoid receptor binding to each peptide on the array, reflected by fluorescent signal, was quantified by image analysis using R-based software (Precision Medicine Lab). The 70 coregulator peptides used represent a range of coregulator nuclear receptor-boxes known to interact with the glucocorticoid receptor and other nuclear receptors. The peptide names, cofactor peptide sequences, uniprot accession and geneID are shown in table 1. The binding profiles were generated for glucocorticoid receptor-LBD treated with the repurposing drugs vs cortisol. An example binding profile for coregulator peptide no 22 with name NCOA1_1421_1444 is shown in FIG. 2 which shows that adapalene, dexamethasone as well as the positive control (PC), cortisol, shows increased binding to coregulator peptide no 22 compared to the negative control (NC), DMSO. The binding profiles for a set of 20 cofactor peptides described in table 2 for each of the tested repurposing drugs are shown in FIG. 3. In each panel the binding profile of the positive control, cortisol is shown as a grey line. The log fold change of binding for coregulator peptide no 22 with name NCOA1_1421_1444 versus the binding to the positive control for the same peptide for each of the tested repurposing drugs is shown in FIG. 4. The log fold change of binding for 20 coregulator peptides described in table 2 versus the binding to the positive control for the same 20 coregulator peptide for each of the tested repurposing drugs is shown in FIG. 5 and FIG. 6. In table 6 the log fold change for the 70 cofactor peptides is shown for each of the tested repurposing drugs versus the positive control, cortisol.

### Example 2

In this example the log fold change results of example 1 were used to determine the similarities between the tested repositioning drugs versus cortisol using the coefficient of determination, R2, between the log fold change data of the tested repurposing drug with the positive control, cortisol using the 70 cofactor peptides. The R2 is calculated using the data for each of the 70 cofactor peptides using the same weight. The similarity index based on the R2 calculation showed that dexamethasone had the highest similarity compared to cortisol followed by adapalene and followed by remdesivir and chloroquine which showed minimal to no similarity with cortisol (table 10, No weight).

### Example 3

In this example a weight factor was applied on the log fold change data of example 1 to determine the similarities between the tested repositioning drugs versus cortisol using the coefficient of determination, R2, between the weighted log fold change data of the tested repurposing drug to the positive control, cortisol. The 8 weight factors are outlined in table 7 thereby favor the higher log fold change values with a weight factor of 1 at the highest absolute log fold change range of 1.75-1.99 and a weight factor of 0.5 at the absolute log fold change range of 1.50-1.74 and a weight factor of 0.25 at the absolute log fold change range of 1.25-1.49 and a weight factor of 0,125 at the absolute log fold change range of 1.00-1.24 and a weight factor of 0.0625 at the absolute log fold change range of 0.75-0.99 and a weight factor of 0.03125 at the absolute log fold change range of 0.50-0.74 and a weight factor of 0 at the remaining absolute lower log fold change ranges. In this example 14 cofactor peptides were used (table 3). The similarity index based on the R2 calculation showed that dexamethasone and adapalene had a similar similarity compared to cortisol followed chloroquine and followed by remdesivir (table 10, Weight-A).

### Example 4

In this example an empirical subset of 4 cofactor peptides were used (table 4) and a weight factor of 1 to differentiate chloroquine from remdesivir based on their R2 or similarity scores. The similarity index based on the R2 calculation showed that dexamethasone and adapalene had a similar high similarity compared to cortisol followed chloroquine which had a higher similarity score compared to the similarity score (R2) obtained in example 1 and the weighted similarity score of example 3 with remdesivir having a negative similarity score (R2). Using such an approach (or a class prediction based assessment) in a drug repositioning program, compounds or drugs can be selected which have a certain log fold change index profile which not necessarily needs to be equal to the positive control or any of the know natural nuclear receptor ligand for the tested nuclear receptor.

### SEQUENCE LISTING

Provided herewith is a Sequence Listing identified by the name of the XML file: “PML-003_Sequence_Listing_20240112.xml” with a creation date of Jan. 12, 2023 and a size of the XML file of 62,112 bytes.

TABLE 1

| ID | Peptide_Name | Peptide_Sequence | Uniprot_Accession | GeneID |
|---|---|---|---|---|
| 1 | ANDR_11_32 | YPRPPSKTYRGAFQNLFQSVRE | P10275 | 367 |
| 2 | CBP_2056_2077 | VQPPRSISPSALQDLLRTLKSP | Q92793 | 1387 |
| 3 | CENPR_159_177 | PHKASRHLDSYEFLKAILN | Q13352 | 23421 |
| 4 | CHD9_1024_1045 | LTGTPLQNTVEELFSLLHFLEP | Q3L8U1 | 80205 |
| 5 | CNOT1_141_162 | LRGFAAQFIKQKLPDLLRSYID | A5YKK6 | 23019 |
| 6 | CNOT1_558_579 | SRILDVAQDLKALSMLLNGTPF | A5YKK6 | 23019 |
| 7 | DHX30_49_70 | EFPQPKNLLNSVIGRALGISHA | Q7L2E3 | 22907 |
| 8 | EP300_2040_2061 | PLKPGTVSQQALQNLLRTLRSP | Q09472 | 2033 |
| 9 | EP300_70_91 | MVQDAASKHKQLSELLRSGSSP | Q09472 | 2033 |
| 10 | GNAQ_22_43 | QLRRDKRDARRELKLLLLGTGE | P50148 | 2776 |
| 11 | IKBB_277_299 | LGSAMLRPNPILARLLRAHGAP | Q15653 | 4793 |
| 12 | JHD2C_2055_2076 | LVSQNNEQGSTLRDLLTTTAGK | Q15652 | 221037 |
| 13 | KMT2D_2674_2695 | QTELEKQRQRQRLRELLIRQQI | O14686 | 8085 |
| 14 | LCOR_41_62 | TSPTAATTQNPVLSKLLMADQD | Q96JN0 | 84458 |
| 15 | MAPE_250_271 | AKFSPYLGQMINLRRLLLSHIH | P78395 | 23532 |
| 16 | MAPE_301_322 | LYVDSLFFLRGRLDQLLRHVMN | P78395 | 23532 |
| 17 | MED1_593_614 | EDFSKVSQNPILTSLLQITGNG | Q15648 | 5469 |
| 18 | MEN1_256_277 | TDSLELLQLQQKLLWLLYDLGH | O00255 | 4221 |
| 19 | MGMT_87_108 | PVFQQESFTRQVLWKLLKVVKF | P16455 | 4255 |
| 20 | MLL2_4176_4197 | LAGPRSEAGHLLLQKLLRAKNV | O14686 | 8085 |
| 21 | MLL2_4703_4724 | RLKKWKGVRWKRLRLLLTIQKG | O14686 | 8085 |
| 22 | NCOA1_1421_1441 | TSGPQTPQAQQKSLLQQLLTE | Q15788 | 8648 |
| 23 | NCOA1_679_700 | SHSSLTERHKILHRLLQEGSPS | Q15788 | 8648 |
| 24 | NCOA1_738_759 | SKKKESKDHQLLRYLLDKDEKD | Q15788 | 8648 |
| 25 | NCOA2_630_651 | SRLHDSKGQTKLLQLLTTKSDQ | Q15596 | 10499 |
| 26 | NCOA2_679_700 | HGTSLKEKHKILHRLLQDSSSP | Q15596 | 10499 |
| 27 | NCOA2_734_755 | PVSPKKKENALLRYLLDKDDTK | Q15596 | 10499 |
| 28 | NCOA2_867_888 | QSTFNNPRPGQLGRLLPNQNLP | Q15596 | 10499 |
| 29 | NCOA3_610_631 | RGPLESKGHKKLLQLLTCSSDD | Q9Y6Q9 | 8202 |
| 30 | NCOA3_674_695 | HGSLLQEKHRILHKLLQNGNSP | Q9Y6Q9 | 8202 |
| 31 | NCOA3_726_747 | QLSPKKKENNALLRYLLDRDDP | Q9Y6Q9 | 8202 |
| 32 | NCOA4_316_337 | KPENGSRETSEKFKLLFQSYNV | Q13772 | 8031 |
| 33 | NCOA6_1480_1501 | VSPAMREAPTSLSQLLDNSGAP | Q14686 | 23054 |
| 34 | NCOR1_1925_1946 | TTITAANFIDVIITRQIASDKD | O75376 | 9611 |
| 35 | NCOR1_2040_2061 | GQVPRTHRLITLADHICQIITQ | O75376 | 9611 |
| 36 | NELFB_329_350 | ETLPRDSPDLLLLLRLLALGQG | Q8WX92 | 25920 |
| 37 | NROB1_138_159 | DHPRQGSILYSLLTSSKQTHVA | P51843 | 190 |
| 38 | NROB1_2_23 | AGENHQWQGSILYNMLMSAKQT | P51843 | 190 |
| 39 | NROB2_107_128 | FEVAEAPVPSILKKILLEEPSS | Q15466 | 8431 |
| 40 | NRIP1_1056_1077 | KDSPRLTKTNPILYYMLQKGGN | P48552 | 8204 |
| 41 | NRIP1_174_195 | DLRCYGVASSHLKTLLKKSKVK | P48552 | 8204 |
| 42 | NRIP1_369_390 | NNIKQAANNSLLLHLLKSQTIP | P48552 | 8204 |
| 43 | NRIP1_489_510 | NSKLNSHQKVTLLQLLLGHKNE | P48552 | 8204 |
| 44 | NRIP1_701_722 | SEIENLLERRTVLQLLLGNPNK | P48552 | 8204 |
| 45 | NRIP1_810_831 | DFSFSKNGLLSRLLRQNQDSYL | P48552 | 8204 |
| 46 | NRIP1_925_946 | SWARESKSFNVLKQLLLSENCV | P48552 | 8204 |
| 47 | NSD1_895_916 | SQNHIPIEPDYKFSTLLMMLKD | Q96L73 | 64324 |
| 48 | PAGR1_103_124 | QPWMPPPSEIQRLYELLAAHGT | Q9BTK6 | 79447 |
| 49 | PAK6_249_270 | PKTRESSLKRRLFRSMFLSTAA | Q9NQU5 | 56924 |
| 50 | PCAF_179_200 | EDADTKQVYFYLFKLLRKSILQ | Q92831 | 8850 |
| 51 | PELP1_169_190 | FRDISMNHLPGLLTSLLGLRPE | Q8IZL8 | 27043 |
| 52 | PELP1_21_42 | TGGLSAVSSGPRLRLLLLESVS | Q8IZL8 | 27043 |
| 53 | PELP1_447_468 | GMLQGGASGEALLTHLLSDISP | Q8IZL8 | 27043 |
| 54 | PIAS2_7_28 | LRNMVSSFRVSELQVLLGFAGR | 075928 | 9063 |
| 55 | PNRC1_306_327 | ENSNQNRELMAVHLKTLLKVQT | Q12796 | 10957 |
| 56 | PPRC1_152_173 | SELLVSPREGSSLHKLLTLSRT | Q5VV67 | 23082 |
| 57 | PR285_1106_1127 | QARLYENLPPAALRKLLRAEPE | Q9BYK8 | 85441 |
| 58 | PRGC1_134_154 | PPQEAEEPSLLKKLLLAPANT | Q9UBK2 | 10891 |
| 59 | PRGC2_146_166 | PAPEVDELSLLQKLLLATSYP | Q86YN6 | 133522 |
| 60 | PRGC2_338_358 | AEFSILRELLAQDVLCDVSKP | Q86YN6 | 133522 |
| 61 | PROX1_58_79 | VVQHADGEKSNVLRKLLKRANS | Q92786 | 5629 |
| 62 | RAD9A_349_370 | PSTVPGTPPPKKFRSLFFGSIL | Q99638 | 5883 |
| 63 | TIF1A_748_769 | SRPQNANYPRSILTSLLLNSSQ | O15164 | 8805 |
| 64 | TIP60_477_498 | SEDIVDGHERAMLKRLLRIDSK | Q92993 | 10524 |
| 65 | TREF1_169_190 | QSAVMDGAPDSALRQLLSQKPM | Q96PN7 | 55809 |
| 66 | TRRAP_972_993 | VAMMSLEDNKHALYQLLAHPNF | Q9Y4A5 | 8295 |
| 67 | TRXR1_133_154 | HGPTLKAYQEGRLQKLLKMNGP | Q16881 | 7296 |
| 68 | UBE3A_650_671 | DLGDSHPVLYQSLKDLLEYEGN | Q05086 | 7337 |
| 69 | WIPI1_120_141 | SIYIHNIKDMKLLKTLLDIPAN | Q5MNZ9 | 55062 |
| 70 | ZNHI3_90_111 | QNLKNLGESATLRSLLLNPHLR | Q15649 | 9326 |

TABLE 2

| ID | Peptide_Name | Peptide_Sequence | Uniprot_Accession | GeneID |
|---|---|---|---|---|
| 2 | CBP_2056_2077 | VQPPRSISPSALQDLLRTLKSP | Q92793 | 1387 |
| 11 | IKBB_278_299 | LGSAMLRPNPILARLLRAHGAP | Q15653 | 4793 |
| 13 | KMT2D_2674_2695 | QTELEKQRQRQRLRELLIRQQI | O14686 | 8085 |
| 15 | MAPE_250_271 | AKFSPYLGQMINLRRLLLSHIH | P78395 | 23532 |
| 20 | MLL2_4176_4197 | LAGPRSEAGHLLLQKLLRAKNV | O14686 | 8085 |
| 21 | MLL2_4703_4724 | RLKKWKGVRWKRLRLLLTIQKG | O14686 | 8085 |
| 22 | NCOA1_1421_1441 | TSGPQTPQAQQKSLLQQLLTE | Q15788 | 8648 |
| 24 | NCOA1_738_759 | SKKKESKDHQLLRYLLDKDEKD | Q15788 | 8648 |
| 26 | NCOA2_679_700 | HGTSLKEKHKILHRLLQDSSSP | Q15596 | 10499 |
| 27 | NCOA2_734_755 | PVSPKKKENALLRYLLDKDDTK | Q15596 | 10499 |
| 31 | NCOA3_726_747 | QLSPKKKENNALLRYLLDRDDP | Q9Y6Q9 | 8202 |
| 39 | NROB2_107_128 | FEVAEAPVPSILKKILLEEPSS | Q15466 | 8431 |
| 42 | NRIP1_369_390 | NNIKQAANNSLLLHLLKSQTIP | P48552 | 8204 |
| 45 | NRIP1_810_831 | DFSFSKNGLLSRLLRQNQDSYL | P48552 | 8204 |
| 52 | PELP1_21_42 | TGGLSAVSSGPRLRLLLLESVS | Q8IZL8 | 27043 |
| 58 | PRGC1_134_154 | PPQEAEEPSLLKKLLLAPANT | Q9UBK2 | 10891 |
| 59 | PRGC2_146_166 | PAPEVDELSLLQKLLLATSYP | Q86YN6 | 133522 |
| 63 | TIF1A_748_769 | SRPQNANYPRSILTSLLLNSSQ | O15164 | 8805 |
| 68 | UBE3A_650_671 | DLGDSHPVLYQSLKDLLEYEGN | Q05086 | 7337 |
| 70 | ZNHI3_90_111 | QNLKNLGESATLRSLLLNPHLR | Q15649 | 9326 |

TABLE 3

| ID | Peptide_Name | Peptide_Sequence | Uniprot_Accession | GeneID |
|---|---|---|---|---|
| 22 | NCOA1_1421_1441 | EDADTKQVYFYLFKLLRKSILQ | Q92831 | 8850 |
| 24 | NCOA1_738_759 | SKKKESKDHQLLRYLLDKDEKD | Q15788 | 8648 |
| 26 | NCOA2_679_700 | HGTSLKEKHKILHRLLQDSSSP | Q15596 | 10499 |
| 27 | NCOA2_734_755 | PVSPKKKENALLRYLLDKDDTK | Q15596 | 10499 |
| 31 | NCOA3_726_747 | QLSPKKKENNALLRYLLDRDDP | Q9Y6Q9 | 8202 |
| 39 | NROB2_107_128 | FEVAEAPVPSILKKILLEEPSS | Q15466 | 8431 |
| 42 | NRIP1_369_390 | NNIKQAANNSLLLHLLKSQTIP | P48552 | 8204 |
| 45 | NRIP1_810_831 | DFSFSKNGLLSRLLRQNQDSYL | P48552 | 8204 |
| 52 | PELP1_21_42 | TGGLSAVSSGPRLRLLLLESVS | Q8IZL8 | 27043 |
| 58 | PRGC1_134_154 | PPQEAEEPSLLKKLLLAPANT | Q9UBK2 | 10891 |
| 59 | PRGC2_146_166 | PAPEVDELSLLQKLLLATSYP | Q86YN6 | 133522 |
| 63 | TIF1A_748_769 | SRPQNANYPRSILTSLLLNSSQ | O15164 | 8805 |
| 68 | UBE3A_650_671 | DLGDSHPVLYQSLKDLLEYEGN | Q05086 | 7337 |
| 70 | ZNHI3_90_111 | QNLKNLGESATLRSLLLNPHLR | Q15649 | 9326 |

TABLE 4

| ID | Peptide_Name | Peptide_Sequence | Uniprot_Accession | GeneID |
|---|---|---|---|---|
| 11 | IKBB_278_299 | LGSAMLRPNPILARLLRAHGAP | Q15653 | 4793 |
| 27 | NCOA2_734_755 | PVSPKKKENALLRYLLDKDDTK | Q15596 | 10499 |
| 59 | PRGC2_146_166 | PAPEVDELSLLQKLLLATSYP | Q86YN6 | 133522 |
| 68 | UBE3A_650_671 | DLGDSHPVLYQSLKDLLEYEGN | Q05086 | 7337 |

TABLE 5

| Drug ID | Drug name | Cas Number |
|---|---|---|
| PC | Cortisol | 50-23-7 |
| 1 | Adapalene | 106685-40-9 |
| 2 | Chloroquine | 54-05-7 |
| 3 | Dexamethasone | 200-003-9 |
| 4 | Remdesivir | 1809249-37-3 |

TABLE 6

| ID | Peptide_name | PC | 1 | 2 | 3 | 5 |
|---|---|---|---|---|---|---|
| 1 | ANDR_11_32 | 0.549 | 0.187 | 0.207 | 0.201 | 0.380 |
| 2 | CBP_2056_2077 | 0.473 | −0.129 | −0.011 | 0.137 | 0.200 |
| 3 | CENPR_159_177 | 0.669 | 0.172 | 0.009 | 0.391 | 0.368 |
| 4 | CHD9_1024_1045 | 0.375 | 0.456 | 0.175 | 0.077 | 0.377 |
| 5 | CNOT1_141_162 | 0.446 | 0.249 | −0.058 | 0.024 | 0.123 |
| 6 | CNOT1_558_579 | 0.386 | 0.149 | −0.022 | 0.055 | 0.251 |
| 7 | DHX30_49_70 | 0.446 | −0.237 | −0.037 | −0.135 | 0.229 |
| 8 | EP300_2040_2061 | 0.383 | −0.241 | −0.016 | −0.033 | 0.139 |
| 9 | EP300_70_91 | 0.813 | 0.505 | | 0.501 | |
| 10 | GNAQ_22_43 | | 0.633 | 0.082 | 0.041 | 0.133 |
| 11 | IKBB_278_299 | 0.611 | −0.247 | −0.049 | 0.304 | 0.150 |
| 12 | JHD2C_2055_2076 | | 0.663 | | 0.863 | |
| 13 | KMT2D_2674_2695 | 0.589 | −0.188 | 0.033 | 0.231 | 0.206 |
| 14 | LCOR_41_62 | 1.238 | 0.699 | | 1.278 | |
| 15 | MAPE_250_271 | 0.090 | 0.394 | 0.016 | 0.159 | 0.219 |
| 16 | MAPE_301_322 | −0.294 | 0.545 | −0.040 | −0.090 | 0.117 |
| 17 | MED1_593_614 | 0.949 | 0.342 | | 1.055 | |
| 18 | MEN1_256_277 | 0.192 | 0.239 | −0.077 | −0.105 | 0.139 |
| 19 | MGMT_87_108 | 0.250 | −0.233 | −0.052 | −0.064 | 0.148 |
| 20 | MLL2_4176_4197 | 0.650 | −0.280 | 0.059 | 0.240 | 0.197 |
| 21 | MLL2_4703_4724 | 0.531 | 0.182 | 0.019 | −0.067 | 0.174 |
| 22 | NCOA1_1421_1441 | 1.715 | 1.193 | | 1.873 | |
| 23 | NCOA1_679_700 | 1.097 | 0.491 | | 0.807 | |
| 24 | NCOA1_738_759 | 1.340 | 0.753 | | 1.213 | 0.275 |
| 25 | NCOA2_630_651 | 1.253 | 0.716 | 0.283 | 1.117 | 0.239 |
| 26 | NCOA2_679_700 | 1.777 | 0.591 | | 0.667 | |
| 27 | NCOA2_734_755 | 1.465 | 0.934 | 0.123 | 1.258 | 0.174 |
| 28 | NCOA2_867_888 | 0.263 | 0.274 | 0.120 | 0.051 | 0.314 |
| 29 | NCOA3_610_631 | 1.176 | 0.613 | 0.615 | 0.989 | 0.422 |
| 30 | NCOA3_674_695 | 0.197 | 0.519 | 0.209 | 0.298 | 0.240 |
| 31 | NCOA3_726_747 | 1.029 | 1.130 | 0.000 | 1.248 | 0.389 |
| 32 | NCOA4_316_337 | 0.839 | 0.672 | 0.275 | 0.681 | 0.566 |
| 33 | NCOA6_1480_1501 | | | | | |
| 34 | NCOR1_1925_1946 | 0.398 | 0.544 | | | |
| 35 | NCOR1_2040_2061 | 0.391 | −0.101 | −0.173 | 0.013 | 0.184 |
| 36 | NELFB_329_350 | 0.195 | −0.017 | −0.038 | −0.083 | 0.134 |
| 37 | NR0B1_138_159 | | 0.716 | 0.247 | 1.261 | 0.162 |
| 38 | NR0B1_2_23 | 0.415 | 0.643 | | 1.007 | |
| 39 | NR0B2_107_128 | 1.538 | 0.929 | | 1.553 | |
| 40 | NRIP1_1056_1077 | | 0.763 | 0.350 | 0.655 | 0.419 |
| 41 | NRIP1_174_195 | 0.213 | 0.252 | 0.045 | 0.069 | −0.784 |
| 42 | NRIP1_369_390 | 1.027 | 0.474 | −0.001 | 0.765 | −0.283 |
| 43 | NRIP1_489_510 | 0.954 | 0.851 | 0.070 | 0.447 | 0.298 |
| 44 | NRIP1_701_722 | 0.609 | −0.257 | 0.204 | 0.573 | 0.539 |
| 45 | NRIP1_810_831 | 0.909 | 0.271 | −0.044 | 0.785 | 0.269 |
| 46 | NRIP1_925_946 | 0.970 | 0.125 | −0.015 | 0.799 | −0.020 |
| 47 | NSD1_895_916 | 0.886 | 0.415 | | 1.173 | |
| 48 | PAGR1_103_124 | 1.057 | 0.301 | | 0.835 | |
| 49 | PAK6_249_270 | 0.362 | 0.112 | 0.034 | −0.146 | 0.271 |
| 50 | PCAF_179_200 | 0.771 | 0.568 | −0.087 | −0.266 | 0.147 |
| 51 | PELP1_169_190 | 0.496 | 0.091 | 0.055 | 0.197 | 0.232 |
| 52 | PELP1_21_42 | 0.888 | 0.504 | −0.041 | 0.397 | 0.242 |
| 53 | PELP1_447_468 | 0.771 | 0.362 | | 0.756 | |
| 54 | PIAS2_7_28 | 0.383 | −0.060 | −0.185 | −0.105 | 0.064 |
| 55 | PNRC1_306_327 | 0.568 | 0.591 | 0.343 | 0.753 | 0.114 |
| 56 | PPRC1_152_173 | 0.380 | 0.146 | −0.160 | 0.753 | 0.085 |
| 57 | PR285_1106_1127 | 1.167 | 0.491 | | 1.254 | 0.579 |
| 58 | PRGC1_134_154 | 1.669 | 1.173 | | 1.804 | |
| 59 | PRGC2_146_166 | 0.902 | 0.388 | 0.099 | 0.799 | 0.272 |
| 60 | PRGC2_338_358 | 1.230 | 0.799 | −0.540 | 0.694 | −0.540 |
| 61 | PROX1_58_79 | 0.547 | 0.337 | 0.276 | 0.403 | 0.296 |
| 62 | RAD9A_349_370 | 0.503 | 0.197 | 0.066 | 0.116 | 0.146 |
| 63 | TIF1A_748_769 | 0.930 | −0.191 | 0.008 | 0.810 | 0.184 |
| 64 | TIP60_477_498 | 0.672 | 0.653 | 0.404 | 0.765 | 0.179 |
| 65 | TREF1_169_190 | 0.886 | 0.602 | | 1.012 | |
| 66 | TRRAP_972_993 | 1.371 | 0.255 | | 1.157 | |
| 67 | TRXR1_133_154 | 0.623 | 0.322 | 0.449 | 0.854 | 0.097 |
| 68 | UBE3A_650_671 | 1.430 | 0.690 | 0.151 | 1.433 | 0.068 |
| 69 | WIPI1_120_141 | 1.049 | 0.556 | | 1.084 | 0.498 |
| 70 | ZNHI3_90_111 | 0.814 | −0.084 | 0.043 | 0.586 | 0.197 |

TABLE 7

| LFC range: | Weight |
|---|---|
| (0-0.24) | — |
| (0.25-0.49) | — |
| (0.5-0.74) | 0.031 |
| (0.75-0.99) | 0.063 |
| (1-1.24) | 0.125 |
| (1.25-1.49) | 0.250 |
| (1.5-1.74) | 0.500 |
| (1.75-1.99) | 1.000 |

TABLE 8

| Peptide_ID | Peptide_name | PC | 1 | 2 | 3 | 4 |
|---|---|---|---|---|---|---|
| 22 | NCOA1_1421_1441 | 1.715 | 1.193 | | 1.873 | |
| 24 | NCOA1_738_759 | 0.168 | 0.094 | | 0.152 | 0.034 |
| 26 | NCOA2_679_700 | 0.056 | 0.018 | | 0.021 | |
| 27 | NCOA2_734_755 | 0.366 | 0.234 | 0.031 | 0.314 | 0.044 |
| 31 | NCOA3_726_747 | 0.515 | 0.565 | 0.000 | 0.624 | 0.194 |
| 39 | NR0B2_107_128 | 0.769 | 0.465 | | 0.777 | |
| 42 | NRIP1_369_390 | 0.128 | 0.059 | 0.000 | 0.096 | −0.035 |
| 45 | NRIP1_810_831 | 0.057 | 0.017 | −0.003 | 0.049 | 0.017 |
| 52 | PELP1_21_42 | 0.028 | 0.016 | −0.001 | 0.012 | 0.008 |
| 58 | PRGC1_134_154 | 0.835 | 0.587 | | 0.902 | |
| 59 | PRGC2_146_166 | 0.113 | 0.048 | 0.012 | 0.100 | 0.034 |
| 63 | TIF1A_748_769 | 0.058 | −0.012 | 0.000 | 0.051 | 0.011 |
| 68 | UBE3A_650_671 | 0.357 | 0.173 | 0.038 | 0.358 | 0.017 |
| 70 | ZNHI3_90_111 | 0.025 | −0.003 | 0.001 | 0.018 | 0.006 |

TABLE 9

| ID | Peptide_Name | PC | 1 | 2 | 3 | 4 |
|---|---|---|---|---|---|---|
| 11 | IKBB_278_299 | 0.611 | 0.247 | 0.049 | 0.304 | 0.150 |
| 27 | NCOA2_734_755 | 1.465 | 0.934 | 0.123 | 1.258 | 0.174 |
| 59 | PRGC2_146_166 | 0.902 | 0.388 | 0.099 | 0.799 | 0.272 |
| 68 | UBE3A_650_671 | 1.430 | 0.690 | 0.151 | 1.433 | 0.068 |

TABLE 10

| Drug ID | No weight | Weight-A | Weight-B |
|---|---|---|---|
| 1 | 0.578 | 0.981 | 0.955 |
| 2 | 0.156 | 0.524 | 0.881 |
| 3 | 0.825 | 0.998 | 0.977 |
| 4 | 0.097 | 0.741 | −0.403 |

### REFERENCES

ⁱIshigami-Yuasa M, Kagechika H. Chemical Screening of Nuclear Receptor Modulators. Int J Moi Sci. 2020 Jul. 31; 21(15):5512. doi: 10.3390/ijms2115551.2. PMID: 327521.36; PMCID: PMC7432305.
ⁱⁱDe Bosscher K, Desniet S J, Clarisse D, Estébanez-Perpiña E, Brunsveld L. Nuclear receptor crosstalk defining the mechanisms for therapeutic innovation. Nat Rev Endocrinol. 2020 Ju1; 16(7):363-377, doi: 10.1038/s41574-020-0349-5, Epub 2020 Apr. 17. PMID: 32303708.
ⁱⁱⁱMcKenna N J, Cooney A J, DeMayo E J, Downes M, Glass C K, Lanz R B, Lazar M A, Mangelsdorf D J, Moore D D, Qin J, Steffen D L, Tsai M J, Tsai S Y, Yu R, Margolis R N, Evans R M, O'Malley B W. Minireview: Evolution of NURSA, the Nuclear Receptor Signaling Atlas. Mol Endocrinol. 2009 June; 23(6):740-6. doi: 10.1210/me.2009-0135. Epub 2009 May 7. Erratum in: Mol Endocrinol. 2009 September; 23(9):1522. PMID: 19423650; PMCM: PMC2691684.
^{iv}Becnel L B, Darlington Y E, Ochsner S A, Easton-Marks J R, Watkins C M, McOwiti A, Kankanamge W H, Wise M W, DeHart M, Margolis R N, McKenna N J. Nuclear Receptor Signaling Atlas: Opening Access to the Biology of Nuclear Receptor Signaling Pathways. PLoS One. 2015 Sep. 1; 10(9):e0135615. doi: 10.1371/journal.pone.0135615. PMID: 26325041; PMCID: PMC4556694.
^{v}Mangelsdorf D J, Evans R M. The RXR heterodimers and orphan receptors. Cell. 1995 Dec. 15; 83(6):841-50. doi: 10.1016/0092-8674(95)90200-7. PMID: 8521508,
^{vi}Rosenfeld M G, Lunyak V V, Glass C K. Sensors and signals: a coactivator/corepressor/epigenetic code for integrating signal-dependent programs of transcriptional response. Genes Dev. 2006 Jun. 1; 20(11):1405-28. doi: 10.1101/gad.1.424806. PMID: 16751179.
^{vii}Spiegelman B M, Heinrich R. Biological control through regulated transcriptional coactivators. Cell. 2004 Oct. 15; 119(2):157-67. doi: 10.1016/j.cell.2004.09.037. PMID: 15479634,
^{viii}Fuentes-Prior P, Rojas A, Hagler A T, Estébanez-Perpiñá E. Diversity of Quaternary Structures Regulates Nuclear Receptor Activities. Trends Biochem Sci. 2019 January; 44(1):2-6. doi: 10.1016/j.tibs.201.8.09.005. Epub 2018 Oct. 4. PMID: 30293659.
^{ix}Hehen C, KleTkhofs S, Clinckemalie L, Spans L, Laurent M, Boonen S, Vanderschueren D, Claessens F. Structural basis for nuclear hormone receptor DNA binding. Mol Cell Endocrinol. 2012 Jan. 30:348(2); 411-7. doi: 10.1016/j.mce.2011.07.025. Epub 2011 Jul. 27. PMID; 21801809.
^{x}Rastinejad F, Huang P, Chandra V, Khorasanizadeh S. Understanding nuclear receptor form and function using structural biology. J Mol Endocrinol, 2013 Nov. 7; 51(3):T1-T21. doi: 10.1530/JME-13-0173. PMID: 24103914; PMCID: PMC: 3571.882.
^{xi}Bilias 1, Moras D. Allosteric controls of nuclear receptor function in the regulation of transcription, J Mol Biol. 2013 Jul. 10; 425(13):2317-29. doi: 10.1016/j.jmb.2013.03.017. Epub 2013 Mar. 15. PMID; 23499886.
^{xii}Tsai M J, O'Malley B W. Molecular mechanisms of action of steroid/thyroid receptor superfamily members. Annu Rev Biochem. 1994; 63:451-86. doi: 10.1146/annurev.bi.63.070194.002315. PMID: 7979245.
^{xiii}Wu B, Barite. E, De S K, Wei J, Purves A, Pellecchia M. High-Throughput Screening by Nuclear Magnetic Resonance (HTS by NMR) for the Identification of PPIs Antagonists. Curr Top Med Chem. 2015; 15(20):2032-42, doi: 10.2174/1568026615666150519102459. PMID: 25986689; PMCID: PMC4545611.
^{xiv}Hua Y, Shun T Y, Strock C J, Johnston P A. High-content positional biosensor screening assay for compounds to prevent or disrupt androgen receptor and transcriptional intermediary factor 2 protein-protein interactions, Assay Drug Dev Technol. 2014 September; 12(7):395-418. doi: 10.1089/adt.2014.594. PMID: 25181412; PMCID: PMC4146499,
^{xv}Fancher A T, Hua Y, Camarco D P, Close D A, Strock C J, Johnston P A. High-Content Screening Campaign to Identify Compounds That Inhibit or Disrupt Androgen Receptor-Transcriptional Intermediary Factor 2 Protein-Protein Interactions for the Treatment of Prostate Cancer. Assay Drug Dev Technol. 2018 August/September; 16(6):297-319. doi: 10.1089/adt.2018.858. Epub 2018 Aug. 15. PMID: 30:109944; PMCID: PMC6114076.
^{xvi}Newman D J, Cragg G M. Natural products as sources of new drugs over the 30 years from 1981 to 2010.1 Nat Prod. 2012 Mar. 23; 75(3):311-35. doi: 10.1021/np200906s. Epub 2012 Feb. 8, PMID: 22316239; PMCID: PMC3721181.
^{xvii}https://www.chembridge.com/screening_libraries/targeted_libraries/#Nuclear%20Receptor [(accessed on May 16, 2022)];
^{xviii}Sahoo B M, Ravi Kumar B V V, Sruti J, Mahapatra M K, Battik B K, Borah P. Drug Repurposing Strategy (DRS): Emerging Approach to Identify Potential Therapeutics for Treatment of Novel Coronavirus Infection. Front Mol Biosci. 2021 Feb. 26; 8:628144, doi: 10.3389/fmolb.2021.628144. PMID: 33718434; PMCID: PMC7953054.
^{xix}Parvathaneni V, Kulkarni N S, Muth A, Gupta V. Drug repurposing: a promising tool to accelerate the drug discovery process, Drug Discov Today. 2019 October; 24(10):2076-2085.dol: 10.1016/j.drudis.2019.06.014. Epub 2019 Jun. 22. PMID: 31238113.
^{xx}Pushpakom S, Iorio F, Eyers P A, Escott K J, Hopper S, Wells A, Doig A, Guilliams T, Latimer J, McNamee C, Norris A, Sanseau P, Cavalla D, Pirmohamed M. Drug repurposing: progress, challenges and recommendations, Nat Rev Drug Discov. 2019 January; 18(1):41-58. doi: 10.1038/nrd.2018.168. Epub 2018 Oct. 12. PMED: 30310233.
^{xxi}Van Moortel L, Gevaert K, Dc Bosscher K. Improved. Glucocorticoid Receptor Ligands: Fantastic Beasts, but How to Find Them? Front Etadocrinol (Lausanne). 2020 Sep. 24:11:559673. doi: 10.3389/fendo.2020.559673. PMID: 33071974; PMCID: PMC7541956.
^{xxii}Takebe T, Iwai R, Ono S. The Current Status of Drug Discovery and Development as Originated in United States Academia: The Influence of Industrial and Academic Collaboration on Drug Discovery and Development. Clin Trans' Sci. 2018 November; 11(6):597-606, doi: 10.1111/cts.12577. Epub 2018 Jul. 30. PMID: 29940695; PMCID: PMC6226120.
^{xxiii}Desmet S J, Bougarne N, Van Moortel L, Dc Cauwer L, Thommis J, Vuyisteke M, Ratnian D, Houtman R, Tavernier J, De Bosscher K. Compound A influences gene regulation of the Dexamethasone-activated glucocorticoid receptor by alternative cofactor recruitment. Sci Rep. 2017 Aug. 14; 7(1):8063. doi: 10.1038/s41598-017-07941-y. PMID: 28808239; PMCID: PMC5556032.
^{xxiv}Desmet. S J, Dejager L, Clarisse D, Thommis J, Melchers D, Bastiaensen N, Ruijtenbeek R, Beck I M, Libert C, Houtman R, Meijer O C, De Bosscher K. Cofactor profiling of the glucocorticoid receptor from a cellular environment, Methods Mol Biol. 2014; 1204; 83-94. doi: 10.1007/978-1-4939-1346-68, PMID: 25182763,
^{xxv}Desmet S J, Dejager L, Clarisse D, Thommis J, Melchers D, Bastiaensen N, Ruijtenbeek R, Beck I M, Libert C, Houtman R, Meijer O C, De Bosscher K. Cofactor profiling of the glucocorticoid receptor from a cellular environment, Methods Mol Biol. 2014; 1204:83-94. doi: 10.1007/978-1-4939-1346-6_8. PMID: 25182763.
^{xxvi}Achermann J C, Schwabe J, Fairall L, Chatterjee K. Genetic disorders of nuclear receptors. J Clin Invest. 2017; 127(4):1181-1192. doi:10.1172/JCI188892
^{xxvii}Zalachoras 1, Houtman R, Atucha E, Doves R, Tijssen A M, Hu P, Lockey P M, Datsun N A, Belanoff J K, Lucassen P J, Joëls M, de Kloet E R, Roozendaal B, Hunt H, Meijer O C. Differential targeting of brain stress circuits with a selective glucocorticoid receptor modulator. Proc Natl Acad Sci USA. 2013 May 7; 110(19):7910-5. doi: 1.0.1073/pnas.1219411110. Epub 2013 Apr. 23. PMID: 23613579; PMCID: PMC3651427.

1. A method for drug repurposing, the method comprising: selecting, by a drug repurposing computing device, a set of at least one regulatory approved drug and at least one negative control compound and optionally at least one failed drug of which said regulatory approved drug are known to interact with the ligand binding domain of a nuclear receptor said interaction leading to a change in the affinity of said nuclear receptor for binding to a set of at least two peptides derived from cofactor proteins generating cofactor binding ratio value for each peptide in the presence and absence of said regulatory approved drugs and said negative control drugs and optionally said failed drugs targeting said nuclear receptor thereby generating a cofactor binding ratio signature for the collection of tested cofactor peptides from said tested regulatory approved drugs and said negative control drugs and optionally said failed drugs and; associating, by the drug repurposing computing device, the cofactor binding ratio signature using weighted cofactor binding ratio values with the regulatory approved drugs targeting said nuclear receptor; calculating, by the drug repurposing computing device, a probability score of a drug to be repurposed for the selected nuclear receptor based on its measured cofactor binding ratio signature in comparison to said weighted cofactor binding ratio signature from said regulatory approved drugs and said negative control drug and optionally said failed drug.

2. The nuclear receptor of claim 1 wherein said nuclear receptor is a full-length nuclear receptor.

3. The full-length nuclear receptor of claim 2 is derived from a cell lysate.

4. The method for generating a said probability score by said drug repurposing computing device according to claim 1 wherein at least two nuclear receptors are used.

5. The nuclear receptors of claim 4 are selected from the list: NR0A1, NR0B1, NR0B2, NR1A1, NR1A2, NR1B1, NR1B2, NR1B3, NR1C1, NR1C2, NR1C3, NR1D1, NR1D2, NR1E1, NR1F1, NR1F2, NR1F3, NR1G1, NR1H1, NR1H2, NR1H3, NR1H4, NR1H5, NR1I1, NR1I2, NR1I3, NR1J1, NR1J2, NR1J3, NR1K1, NR2A1, NR2A2, NR2B1, NR2B2, NR2B3, NR2B4, NR2C1, NR2C2, NR2E1, NR2E3, NR2F1, NR2F2, NR2F6, NR3A1, NR3A2, NR3B1, NR3B2, NR3B3, NR3C1, NR3C2, NR3C3, NR3C4, NR3D, NR3E, NR3F, NR4A1, NR4A2, NR4A3, NR5A1, NR5A2, NR5B1, NR6A1, NR7A1, NR7B1, NR7C1, and NR8A1.

6. The nuclear receptors of claim 4 comprising of a wild-type nuclear receptor variant and one or more mutant nuclear receptor variants

7. The nuclear receptors of claim 4 comprising of a wild-type nuclear receptor variant and one or more nuclear receptor variants each containing one or more post-translational modifications


SEQUENCE LISTING (70 sequences)

SEQ ID NO: 1 - protein, 22 aa, Homo sapiens
YPRPPSKTYRGAFQNLFQSVRE

SEQ ID NO: 2 - protein, 22 aa, Homo sapiens
VQPPRSISPSALQDLLRTLKSP

SEQ ID NO: 3 - protein, 19 aa, Homo sapiens
PHKASRHLDSYEFLKAILN

SEQ ID NO: 4 - protein, 22 aa, Homo sapiens
LTGTPLQNTVEELFSLLHFLEP

SEQ ID NO: 5 - protein, 22 aa, Homo sapiens
LRGFAAQFIKQKLPDLLRSYID

SEQ ID NO: 6 - protein, 22 aa, Homo sapiens
SRILDVAQDLKALSMLLNGTPF

SEQ ID NO: 7 - protein, 22 aa, Homo sapiens
EFPQPKNLLNSVIGRALGISHA

SEQ ID NO: 8 - protein, 22 aa, Homo sapiens
PLKPGTVSQQALQNLLRTLRSP

SEQ ID NO: 9 - protein, 22 aa, Homo sapiens
MVQDAASKHKQLSELLRSGSSP

SEQ ID NO: 10 - protein, 22 aa, Homo sapiens
QLRRDKRDARRELKLLLLGTGE

SEQ ID NO: 11 - protein, 22 aa, Homo sapiens
LGSAMLRPNPILARLLRAHGAP

SEQ ID NO: 12 - protein, 22 aa, Homo sapiens
LVSQNNEQGSTLRDLLTTTAGK

SEQ ID NO: 13 - protein, 22 aa, Homo sapiens
QTELEKQRQRQRLRELLIRQQI

SEQ ID NO: 14 - protein, 22 aa, Homo sapiens
TSPTAATTQNPVLSKLLMADQD

SEQ ID NO: 15 - protein, 22 aa, Homo sapiens
AKFSPYLGQMINLRRLLLSHIH

SEQ ID NO: 16 - protein, 22 aa, Homo sapiens
LYVDSLFFLRGRLDQLLRHVMN

SEQ ID NO: 17 - protein, 22 aa, Homo sapiens
EDFSKVSQNPILTSLLQITGNG

SEQ ID NO: 18 - protein, 22 aa, Homo sapiens
TDSLELLQLQQKLLWLLYDLGH

SEQ ID NO: 19 - protein, 22 aa, Homo sapiens
PVFQQESFTRQVLWKLLKVVKF

SEQ ID NO: 20 - protein, 22 aa, Homo sapiens
LAGPRSEAGHLLLQKLLRAKNV

SEQ ID NO: 21 - protein, 22 aa, Homo sapiens
RLKKWKGVRWKRLRLLLTIQKG

SEQ ID NO: 22 - protein, 21 aa, Homo sapiens
TSGPQTPQAQQKSLLQQLLTE

SEQ ID NO: 23 - protein, 22 aa, Homo sapiens
SHSSLTERHKILHRLLQEGSPS

SEQ ID NO: 24 - protein, 22 aa, Homo sapiens
SKKKESKDHQLLRYLLDKDEKD

SEQ ID NO: 25 - protein, 22 aa, Homo sapiens
SRLHDSKGQTKLLQLLTTKSDQ

SEQ ID NO: 26 - protein, 22 aa, Homo sapiens
HGTSLKEKHKILHRLLQDSSSP

SEQ ID NO: 27 - protein, 22 aa, Homo sapiens
PVSPKKKENALLRYLLDKDDTK

SEQ ID NO: 28 - protein, 22 aa, Homo sapiens
QSTFNNPRPGQLGRLLPNQNLP

SEQ ID NO: 29 - protein, 22 aa, Homo sapiens
RGPLESKGHKKLLQLLTCSSDD

SEQ ID NO: 30 - protein, 22 aa, Homo sapiens
HGSLLQEKHRILHKLLQNGNSP

SEQ ID NO: 31 - protein, 22 aa, Homo sapiens
QLSPKKKENNALLRYLLDRDDP

SEQ ID NO: 32 - protein, 22 aa, Homo sapiens
KPENGSRETSEKFKLLFQSYNV

SEQ ID NO: 33 - protein, 22 aa, Homo sapiens
VSPAMREAPTSLSQLLDNSGAP

SEQ ID NO: 34 - protein, 22 aa, Homo sapiens
TTITAANFIDVIITRQIASDKD

SEQ ID NO: 35 - protein, 22 aa, Homo sapiens
GQVPRTHRLITLADHICQIITQ

SEQ ID NO: 36 - protein, 22 aa, Homo sapiens
ETLPRDSPDLLLLLRLLALGQG

SEQ ID NO: 37 - protein, 22 aa, Homo sapiens
DHPRQGSILYSLLTSSKQTHVA

SEQ ID NO: 38 - protein, 22 aa, Homo sapiens
AGENHQWQGSILYNMLMSAKQT

SEQ ID NO: 39 - protein, 22 aa, Homo sapiens
FEVAEAPVPSILKKILLEEPSS

SEQ ID NO: 40 - protein, 22 aa, Homo sapiens
KDSPRLTKTNPILYYMLQKGGN

SEQ ID NO: 41 - protein, 22 aa, Homo sapiens
DLRCYGVASSHLKTLLKKSKVK

SEQ ID NO: 42 - protein, 22 aa, Homo sapiens
NNIKQAANNSLLLHLLKSQTIP

SEQ ID NO: 43 - protein, 22 aa, Homo sapiens
NSKLNSHQKVTLLQLLLGHKNE

SEQ ID NO: 44 - protein, 22 aa, Homo sapiens
SEIENLLERRTVLQLLLGNPNK

SEQ ID NO: 45 - protein, 22 aa, Homo sapiens
DFSFSKNGLLSRLLRQNQDSYL

SEQ ID NO: 46 - protein, 22 aa, Homo sapiens
SWARESKSFNVLKQLLLSENCV

SEQ ID NO: 47 - protein, 22 aa, Homo sapiens
SQNHIPIEPDYKFSTLLMMLKD

SEQ ID NO: 48 - protein, 22 aa, Homo sapiens
QPWMPPPSEIQRLYELLAAHGT

SEQ ID NO: 49 - protein, 22 aa, Homo sapiens
PKTRESSLKRRLFRSMFLSTAA

SEQ ID NO: 50 - protein, 22 aa, Homo sapiens
EDADTKQVYFYLFKLLRKSILQ

SEQ ID NO: 51 - protein, 22 aa, Homo sapiens
FRDISMNHLPGLLTSLLGLRPE

SEQ ID NO: 52 - protein, 22 aa, Homo sapiens
TGGLSAVSSGPRLRLLLLESVS

SEQ ID NO: 53 - protein, 22 aa, Homo sapiens
GMLQGGASGEALLTHLLSDISP

SEQ ID NO: 54 - protein, 22 aa, Homo sapiens
LRNMVSSFRVSELQVLLGFAGR

SEQ ID NO: 55 - protein, 22 aa, Homo sapiens
ENSNQNRELMAVHLKTLLKVQT

SEQ ID NO: 56 - protein, 22 aa, Homo sapiens
SELLVSPREGSSLHKLLTLSRT

SEQ ID NO: 57 - protein, 22 aa, Homo sapiens
QARLYENLPPAALRKLLRAEPE

SEQ ID NO: 58 - protein, 21 aa, Homo sapiens
PPQEAEEPSLLKKLLLAPANT

SEQ ID NO: 59 - protein, 21 aa, Homo sapiens
PAPEVDELSLLQKLLLATSYP

SEQ ID NO: 60 - protein, 21 aa, Homo sapiens
AEFSILRELLAQDVLCDVSKP

SEQ ID NO: 61 - protein, 22 aa, Homo sapiens
VVQHADGEKSNVLRKLLKRANS

SEQ ID NO: 62 - protein, 22 aa, Homo sapiens
PSTVPGTPPPKKFRSLFFGSIL

SEQ ID NO: 63 - protein, 22 aa, Homo sapiens
SRPQNANYPRSILTSLLLNSSQ

SEQ ID NO: 64 - protein, 22 aa, Homo sapiens
SEDIVDGHERAMLKRLLRIDSK

SEQ ID NO: 65 - protein, 22 aa, Homo sapiens
QSAVMDGAPDSALRQLLSQKPM

SEQ ID NO: 66 - protein, 22 aa, Homo sapiens
VAMMSLEDNKHALYQLLAHPNF

SEQ ID NO: 67 - protein, 22 aa, Homo sapiens
HGPTLKAYQEGRLQKLLKMNGP

SEQ ID NO: 68 - protein, 22 aa, Homo sapiens
DLGDSHPVLYQSLKDLLEYEGN

SEQ ID NO: 69 - protein, 22 aa, Homo sapiens
SIYIHNIKDMKLLKTLLDIPAN

SEQ ID NO: 70 - protein, 22 aa, Homo sapiens
QNLKNLGESATLRSLLLNPHLR